(19) United States
(12) Patent Application Publication
(10) Pub. No.: US 2025/0311688 A1
(43) Pub. Date: Oct. 9, 2025

(54) METHODS AND SYSTEMS FOR CANNABINOID PRODUCT PRODUCTION

(71) Applicant: Ojai Energetics PBC, Ojai, CA (US)

(72) Inventor: William Kleidon, Ojai, CA (US)

(73) Assignee: Ojai Energetics PBC, Ojai, CA (US)

(21) Appl. No.: 19/241,690

(22) Filed: Jun. 18, 2025

Related U.S. Application Data
(63) Continuation of application No. 17/482,142, filed on Sep. 22, 2021, now abandoned.
Application No. 17/482,142 is a continuation of application No. PCT/US2020/024968, filed on Mar. 26, 2020.
(60) Provisional application No. 62/824,727, filed on Mar. 27, 2019.

(51) Int. Cl. A01H 1/00; A01D 45/00; A01G 7/04; A01H 1/06

(52) U.S. Cl. CPC A01H 1/101; A01D 45/00; A01G 7/045; A01H 1/06

(57) ABSTRACT

Methods and systems for the alteration of cannabinoid expression and composition are described. Modular systems for processing cannabis and hemp are described. Described components of the modular system include improved recovery of high-value cannabis components and methods for utilizing residual biomass components after the cannabis plant has been fully processed.

### CROSS-REFERENCE

This application is a continuation of International Patent Application No. PCT/US2020/024968 filed on Mar. 26, 2020, claims the benefit of U.S. Patent Application No. 62/824,727, filed Mar. 27, 2019, which is entirely incorporated herein by reference.

### BACKGROUND

Cannabinoids are a class of chemicals that can act on cannabinoid receptors. Cannabinoid receptor ligands include endocannabinoids, which can be found naturally occurring in humans and other animals, phytocannabinoids, which can be found in cannabis and other plants, and synthetic cannabinoids. Cannabinoids include tetrahydrocannabinol (THC), such as delta-9-tetrahydrocannabinol, and cannabidiol (CBD). At least 100 different cannabinoids have been isolated from cannabis.

Cannabinoids may be isolated from plants of the Cannabaceae family. In addition to the pharmaceutical applications of cannabinoids that are isolated from plants such as Cannabis sativa and Cannabis indica, cannabis plants may also be an important source of other products, often referred to as hemp products, such as high-strength bast fibers, which have applications in materials such as textiles and building materials.

Cannabis plants may be cultivated, grown, and harvested by traditional agricultural methods. After harvest, a variety of methods may be employed to fully utilize the resources offered by the cannabis plant.

### SUMMARY

The present disclosure provides methods for enhancing the production and utilization of cannabis and hemp plants. Methods and systems may facilitate the increasing, decreasing, or altering of the cannabinoid composition of cannabis plants. These methods may include methods for producing one or more epigenetic modifications in the cannabis plant to alter the chemical composition or yield of expressed cannabinoids. The one or more epigenetic modifications may regulate epigenetic drift in the cannabis plant. Methods and systems may also be described for enhancing the utilization efficiency of cannabis components after harvesting the plant. Methods described may include improved cannabidiol (CBD) extraction processes and the utilization of cannabis components for biofuel generation processes such as biomass gasification, biochar generation, and biomass pelletization.

In an aspect, the present disclosure provides a method of creating at least one epigenetic modification in a Cannabis plant, the method comprising: exposing the Cannabis plant to at least one member selected from the group consisting of: (i) ultraviolet (UV) light; (ii) acoustic energy; (iii) heat; and (iv) a chemical agent, wherein the Cannabis plant is exposed to the at least one member under conditions sufficient to produce the at least one epigenetic modification in at least a portion of a genome of the Cannabis plant.

In some embodiments, the at least one epigenetic modification is characterized by an increased level of expression of one or more cannabinoids in the Cannabis plant. In some embodiments, the at least one epigenetic modification is characterized by an altered composition of cannabinoids expressed in the Cannabis plant. In some embodiments, the at least one epigenetic modification is characterized by an increased level of expression of one or more terpenes in the Cannabis plant. In some embodiments, the at least one epigenetic modification is characterized by an altered composition of terpenes expressed in the Cannabis plant. In some embodiments, the at least one epigenetic modification is characterized by an increased rate of growth of the Cannabis plant.

In some embodiments, the exposing of the Cannabis plant to the at least one member occurs after development of a foliage in the Cannabis plant.

In some embodiments, the Cannabis plant is exposed to at least two members selected from the group consisting of: (i) UV light, (ii) acoustic energy, (iii) heat, and (iv) the chemical agent. In some embodiments, the Cannabis plant is exposed to at least three members selected from the group consisting of (i) UV light, (ii) acoustic energy, (iii) heat, and (iv) the chemical agent. In some embodiments, the Cannabis plant is exposed to (i) UV light, (ii) acoustic energy, (iii) heat, and (iv) the chemical agent.

In another aspect, the present disclosure provides a method of creating at least one epigenetic modification in a plant, the method comprising: exposing the plant to at least one member selected from the group consisting of: (i) ultraviolet (UV) light; (ii) acoustic energy; (iii) heat; and (iv) a chemical agent, wherein the plant is exposed to the at least one member under conditions sufficient to produce the at least one epigenetic modification in at least a portion of a genome of the plant, and wherein the at least one epigenetic modification in the at least the portion of the genome is characterized by (a) an increased level of expression of one or more cannabinoids in the plant, (b) an altered composition of cannabinoids expressed in the plant, (c) an increased level of expression of one or more terpenes in the plant, or (d) an altered composition of terpenes expressed in the plant.

In some embodiments, the method comprises exposing the plant to two or more members selected from the group consisting of: (i) ultraviolet (UV) light; (ii) acoustic energy; (iii) heat; and (iv) a chemical agent. In some embodiments, the method comprises exposing the plant to three or more members selected from the group consisting of: (i) ultraviolet (UV) light; (ii) acoustic energy; (iii) heat; and (iv) a chemical agent. In some embodiments, the method comprises exposing the plant to (i) ultraviolet (UV) light; (ii) acoustic energy; (iii) heat; and (iv) a chemical agent.

In another aspect, the present disclosure provides a Cannabis plant comprising an altered epigenomic profile, wherein the altered epigenomic profile is produced by exposure of the Cannabis plant to at least one member selected from the group consisting of: (i) ultraviolet (UV) light; (ii) acoustic energy; (iii) heat; and (iv) a chemical agent.

In some embodiments, the altered epigenomic profile is produced by exposure of the Cannabis plant to at least two members selected from the group consisting of: (i) UV light, (ii) acoustic energy, (iii) heat, and (iv) the chemical agent. In some embodiments, the altered epigenomic profile is produced by exposure of the Cannabis plant to at least three members selected from the group consisting of: (i) UV light, (ii) acoustic energy, (iii) heat, and (iv) the chemical agent. In some embodiments, the altered epigenomic profile is produced by exposure of the Cannabis plant to (i) UV light, (ii) acoustic energy, (iii) heat, and (iv) the chemical agent.

In another aspect, the present disclosure provides a method for increasing an amount of one or more cannabinoids expressed in a Cannabis plant, the method comprising: (a) providing the Cannabis plant, wherein the Cannabis plant comprises a foliage; and (b) spraying the foliage with a solution, wherein the solution comprises at least one cannabinoid precursor.

In some embodiments, the at least one cannabinoid precursor comprises at least one member selected from the group consisting of: (i) olivetol, (ii) olivetolic acid, (iii) cannabigerol (CBG), and (iv) geranyl pyrophosphate (GPP). In some embodiments, the at least one cannabinoid precursor comprises two or more members selected from the group consisting of: (i) olivetol, (ii) olivetolic acid, (iii) CBG, and (iv) GPP. In some embodiments, the at least one cannabinoid precursor comprises three or more members selected from the group consisting of: (i) olivetol, (ii) olivetolic acid, (iii) CBG, and (iv) GPP. In some embodiments, the at least one cannabinoid precursor comprises: (i) olivetol, (ii) olivetolic acid, (iii) CBG, and (iv) GPP.

In some embodiments, the at least one cannabinoid precursor comprises olivetol. In some embodiments, the at least one cannabinoid precursor comprises olivetolic acid. In some embodiments, the at least one cannabinoid precursor comprises CBG. In some embodiments, the at least one cannabinoid precursor comprises GPP.

In a different aspect, the present disclosure provides a modular system for a continuous processing of a Cannabis plant, the modular system comprising a decortication unit and one or more additional processing units selected from the group consisting of: (i) a foliage removal unit; (ii) a de-gumming unit; (iii) a trichome collection unit; (iv) a seed collection unit; (v) a seed processing unit; (vi) a bast fiber collection unit; (vii) a bast fiber processing unit; (viii) a bundling residual biomass unit; (ix) a cannabinoid extraction unit; (x) a biomass gasification unit; (xi) a biochar production unit; and (xii) a biomass pelletization unit, wherein the decortication unit and the one or more additional processing units are operatively coupled to each other for the continuous processing of the Cannabis plant.

In some embodiments, the modular system comprises two or more of the additional processing units selected from the group consisting of: (i)-(xii). In some embodiments, the modular system comprises three or more of the additional processing units selected from the group consisting of: (i)-(xii). In some embodiments, the modular system comprises four or more of the additional processing units selected from the group consisting of: (i)-(xii). In some embodiments, the modular system comprises five or more of the additional processing units selected from the group consisting of: (i)-(xii). In some embodiments, the modular system comprises six or more of the additional processing units selected from the group consisting of: (i)-(xii). In some embodiments, the modular system comprises seven or more of the additional processing units selected from the group consisting of: (i)-(xii). In some embodiments, the modular system comprises eight or more of the additional processing units selected from the group consisting of: (i)-(xii). In some embodiments, the modular system comprises nine or more of the additional processing units selected from the group consisting of: (i)-(xii). In some embodiments, the modular system comprises eleven or more of the additional processing units selected from the group consisting of: (i)-(xii). In some embodiments, the modular system comprises all of the additional processing units (i)-(xii).

In some embodiments, the decortication unit and the one or more additional processing units are operatively coupled to each other via one or more transport units. In some embodiments, the one or more transport units is selected from the group consisting of: a roller, a belt, a chain, a chute, and a pulley.

In some embodiments, the decortication unit and the one or more additional processing units are releasably coupled to each other.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts possible systems for altering the cannabinoid yield and composition of cannabis plants via controlling epigenetic modification during plant growth and development.

FIG. 2 shows a schematic of a modular cannabis production and processing system.

FIG. 3 shows a computer system that is programmed or otherwise configured to implement methods provided herein.

### DETAILED DESCRIPTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

The present disclosure describes systems and methods for altering the production of plant products, e.g., cannabinoid products. The cannabinoid products described herein may include plants that are rich in one or more cannabinoids, as well as products derived from cannabinoid-rich plants, such as cannabidiol (CBD) oil. The plants may comprise one or more Cannabis plants. The plants may comprise non-Cannabis plants. The present disclosure also provides methods for altering the biological or chemical properties of cannabinoid-rich plants, methods for processing cannabinoid-rich plants in substituent components, and methods for altering extraction processes from cannabinoid-rich plants to create optimized cannabinoid compositions. In some instances, systems and methods may be applied to cannabinoid-deficient plants.

Cannabinoids comprise a class of chemical compounds that bind to the cannabinoid receptor system of many animals, including humans. Cannabinoids may be broadly grouped into categories such as endocannabinoids that are naturally-produced by animals for internal signaling, phytocannabinoids that are produced by plants, and synthetic cannabinoids that are manufactured. Cannabinoids may produce a broad range of pharmacological effects, making them an active target for pharmaceutical research. Most commercially available cannabinoids are derived from plants of the Cannabis genus. At least 100 cannabinoid compounds have been derived from cannabis plants, including such common compounds as tetrahydrocannabinol (THC), cannabinol (CBN), and CBD.

The production of cannabinoid products, such as CBD oil, is a complex multi-stage process involving the growth of cannabis plants, harvesting, processing of the plants into components, extraction of cannabinoids, and formulation of the cannabinoids after extraction. The present disclosure is drawn to particular methods for improving the production of cannabinoid products. Specifically, methods and systems are described that may alter the growth characteristics and growth conditions of Cannabis plants to affect the yield and composition of cannabinoids produced within the plants. Also, methods and systems are described that may improve the post-harvest processing of cannabis plants into substituent components. Methods and systems are also described that may enhance the yield or alter the chemical composition of cannabis-derived cannabinoid compositions during extraction and post-processing.

The present disclosure describes methods and systems for producing a plant with an enhanced yield or composition of cannabinoids. Cannabinoid-producing plants are primarily from the family Cannabaceae (also known as the hemp family), and the division Manoliophyta (the flowering plants). Common species of cannabis plants include Cannabis saliva, Cannabis indica, and Cannabis ruderalis. The systems and methods of the present disclosure may apply to any other cannabinoid-producing plants. Cannabis plants are widely used for the fibrous materials that can be obtained from harvested plants, as well as for unique pharmacological compounds due to the presence of cannabinoids, a group of more than 100 natural products that mainly accumulate in female flowers. Δ⁹-Tetrahydrocannabinol (i.e., “THC”) is the principle psychoactive cannabinoid and the compound responsible for the analgesic, antiemetic and appetite-stimulating effects of cannabis. Cannabinoid content and composition is highly variable among cannabis plants. Selective breeding of cannabis and improved cultivation practices have led to increased potency in the past several decades. This breeding effort has produced hundreds of strains that differ in cannabinoid composition, as well as appearance and growth characteristics.

Cannabis plants contain several important parts with different uses for each, Cannabis plants can typically comprise roots, stalks, stems, leaves, and flowering bodies. Stalks may be utilized for their fibrous content, including a fibrous layer called bast between the outer bark and the woody inner xylem or hurd. Bast fibers extracted from various plants may be used in textiles, clothing, paper, and building materials, Bast fibers may comprise about 5%, 6%, 7%, 8% 9%, 10% 11%, 12%, 13%, 14%, 15%, 16% 17%, 18%, 19%, 20%, 21%, 22%, 23%, 24%, 25%, 26%, 27%, 28%, 29%, 30%, 31%, 32%33%, 34%, 35, 36%, 37%, 38%, 39%, 40% 41%, 42%, 43%, 44%, 45%, 46%, 47%, 48%, 49%, or about 50% of the stalk on a dry-weight basis, Bast fibers may comprise no less than about 5%, 6%, 7% 8%, 9%, 10%, 11%, 12%, 13%, 14%, 15%, 16%, 17%, 18%, 19%, 20%, 21%, 22%, 23%, 24%, 25% 26%, 27%, 28%, 29%, 30%, 31%, 2%, 33%, 34%, 35%, 36%, 37%, 38%, 39%, 40%, 41%, 4)%, 43%, 44%, 45%, 46%, 47% 48%, 49%, or no less than about 50% of the stalk on a dry-weight basis. Bast fibers may comprise no greater than about 50%, 49%, 48%, 47%, 46%, 45%, 44%, 43%, 42, 41%, 40%, 39%, 38%, 37%, 36%, 35% 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, or no greater than about 5% of the stalk on a dry-weight basis, Bast fibers may comprise a, dry-weight percentage of the stalk from about 5% to about 10%, from about 5% to about 15%, from about 5% to about 20%, from about 5% to about 25%, from about 5% to about 30%, from about 5% to about 35%, from about 5% to about 40%, from about 5% to about 45%, from about 5,% to about 50%, from about 10% to about 15%, from about 10% to about 20%, from about 10% to about 25%, from about 10% to about 30%, from about 10% to about 35%, from about 10% to about 40%, from about 10% to about 45%, from about 10% to about 50%, from about 15% to about 20%, from about 15% to about 25%, from about 15% to about 30%, from about 15% to about 35+, from about 15% to about 40%, from about 15% to about 45%, from about 15% to about 50%, from about 20% to about 25%, from about 20% to about 30%, from about 20% to about 35%, from about 20% to about 40%, from about 20% to about 45%, from about 20% to about 50%, from about 25% to about 30%, from about 25% to about 35%, from about 25% to about 40%, from about 25% to about 45%, from about 25% to about 50%, from about 30% to about 35%, from about 30% to about 40%, from about 30% to about 45%, from about 30% to about 50%, from about 35% to about 40%, from about 35% to about 45%, from about 35% to about 50%, from about 40% to about 45%, from about 40% to about 50%, or from about 45% to about 50%. A bark-like covering surrounds the bast fibers and the pectin-containing material around each fiber to form an outer sheath. The breaking down and/or removing of a substantial portion of this outer sheath may be referred to as decortication. The cannabinoid composition of Cannabis stalk material may differ from compositions found in other portions of the plant. The stalk may comprise THC or THC-like cannabinoids at a lower level than other portions of the Cannabis plant. Some cannabis stalks may comprise THC or THC-like cannabinoids at less than about 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%. 0.08%, 0.07%, 0.06%, 0.05%, 0.04%. 0.03%, 0.02%, or about 0.01% of the total cannabinoid composition on a weight basis, Cannabis plants may be selectively bred and grown to enhance or optimize their bast yields.

A Cannabis plant may also comprise a flowering body. The flowering body may comprise several substituent portions such as buds, pistils, and trichomes (also referred to as kief). Trichomes or other flowering body substituents may be collected specifically as a source for deriving cannabinoid compound's. The flowering body may comprise THC or THC-like cannabinoids at a higher level than other portions of the Cannabis plant. Some Cannabis flowering bodies may comprise T-IC or THC-like cannabinoids of at least about 0.01%, 0.02%, 0.03%, 0.04%, 0.05% 0.06%, 0.07%, 0.08%, 0.09%, 0.1%, 0.2%, 0.3%, 0.4%, 0.5%, 0.6%, 0.7%, 0.8%, 0.9%, 1%, 2%, 3% 4%, 5%, 6%, 7%, 8%, 9%, 10%, 15%, or at least about 20% of the total cannabinoid composition on a weight basis. Cannabis plants may be selectively bred and grown to enhance the cannabinoid yield or composition in its flowering bodies. Harvesting processes may specifically target the collection of specific flowering body components such as the trichomes.

Cannabis plants may be grown commercially by several methods. In some aspects, cannabis plants may be cultivated on outdoor plots. In other aspects, cannabis plants may be grown in indoor enclosures such as greenhouses or grow rooms. In some aspects, indoor cannabis cultivation may utilize soil as a growth medium. Indoor agriculture may allow plants to be grown in conditions that are optimized for particular variables, such as temperature, humidity, carbon dioxide (CO₂) level, light spectral range, light intensity, soil composition, soil pH, watering amount, watering frequency, and acoustics, Indoor cultivation systems may deliver water via root infusion systems or misting systems. Indoor agriculture may offer increased protection of cannabis plants to outdoor risks such as fungal infection, insect predation, nematode predation, storm damage, frost damage, and other natural risks. Indoor-grown cannabis plants may mature up to about 5%, 6%, 7%, 8%, 9%, 10%, 11%, 12%, 13%, 14%, 15%, 16%, 17%, 18%, 19%, 20%, 21%, 22%, 23%, 24%, or 25% faster and produce up to 5%, 6%, 7%, 8%, 9%, 10%, 11%, 12%, 13%, 14%, 15% 16%, 17%, 18%, 19%, 20%, 21%, 22%, 23%, 24%, 25%, 26%, 27%, 28%, 29%, or 30% more mass than the same plants grown outdoors. In other aspects, cannabis may be grown hydroponically. Hydroponics may comprise any method of growing plants in a water-based, nutrient rich solution. Hydroponics may not use soil, instead utilizing the root system, which is supported using an inert medium such as perlite, rock wool, clay pellets, peat moss, or vermiculite. Hydroponics agriculture allows the root system to come in direct contact with the nutrient solution, while also having access to oxygen, which is essential for proper growth. Hydroponic cultivation may allow plants to be grown in conditions that are optimized for particular variables, such as temperature, humidity, CO₂ level, light spectral range, light intensity, soil composition, soil pH, watering amount, watering frequency, and acoustics. A hydroponic system may allow an increased rate of growth and increased size in plants. In a hydroponic system, cannabis plants may mature up to about 5%, 6%, 7%, 8%, 9%, 10%, 11%, 12%, 13%, 14%, 15%, 16%, 17%, 18%, 19%, 20%, 21%, 22%, 23%, 24%, or 25% faster and produce up to 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% more mass than the same plants grown in soil.

Indoor cultivation may allow the growth temperature of cannabis plants to be controlled. An optimal growth temperature may be maintained over the entire plant life. An optimal growth temperature may be varied at different growth phases for a particular plant type. The optimal growth temperature may vary depending upon the species. The optimal growth temperature may be at least about 10 degrees Celsius (° C.), 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or at least about 40° C. The optimal growth temperature may be no greater than about 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., or no greater than about 10° C. The optimal growth temperature may occur in a range from about 10° C. to about 13° C., about 10° C. to about 16° C., about 10° C. to about 19° C., about 10° C. to about 22° C., about 10° C. to about 25° C., about 10° C. to about 28° C., about 10° C. to about 31° C., about 10° C. to about 34° C., about 10° C. to about 37° C., about 10° C. to about 40° C., about 13° C. to about 16° C., about 13° C. to about 19° C., about 13° C. to about 22° C., about 13° C. to about 25° C., about 13° C. to about 28° C., about 13° C. to about 31° C., about 13° C. to about 34° C., about 13° C. to about 37° C., about 13° C. to about 40° C., about 16° C. to about 19° C., about 16° C. to about 22° C., about 16° C. to about 25° C., about 16° C. to about 28° C., about 16° C. to about 31° C., about 16° C. to about 34° C., about 16° C. to about 37° C., about 16° C. to about 40° C., about 19° C. to about 22° C., about 19° C. to about 25° C., about 19° C. to about 28° C., about 19° C. to about 31° C., about 19° C. to about 34° C., about 19° C. to about 37° C., about 19° C. to about 40° C., about 22° C. to about 25° C., about 22° C. to about 28° C., about 22° C. to about 31° C., about 22° C. to about 34° C., about 22° C. to about 37° C., about 22° C. to about 40° C., about 25° C. to about 28° C., about 25° C. to about 31° C., about 25° C. to about 34° C., about 25° C. to about 37° C., about 25° C. to about 40° C., about 28° C. to about 31° C., about 28° C. to about 34° C., about 28° C. to about 37° C., about 28° C. to about 40° C., about 31° C. to about 34° C., about 31° C. to about 37° C., about 31° C. to about 40° C., about 34° C. to about 37° C., about 34° C. to about 40° C., or about 37° C. to about 40° C.,

Indoor cultivation may allow for optimized humidity control during the growth of Cannabis plants. An optimal growth humidity may be maintained over the entire plant life. An optimal growth humidity may be varied at different growth phases for a particular plant type. The optimal growth humidity may vary depending upon the species. The optimal growth humidity may be at least about 1%, 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 99%, or at least about 100%. The optimal growth humidity may be no more than about 100%, 99%, 98%, 96%, 94%, 92%, 90%, 88%, 86%, 84%, 82%, 80%, 78%, 76%, 74%, 72%, 70%, 68%, 66%, 64%, 62%, 60%, 58%, 56%, 54%, 52%, 50%, 48%, 46%, 44%, 42%, 40%, 38%, 36%, 34%, 32%, 30%, 28%, 26%, 24%, 22%, 20%, 18%, 16%, 14%, 12%, 90%, 8%, 6%, 4%, 2%, or no more than about 1%. The optimal growth humidity may occur in a range from about 1% to about 2%, about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 1% to about 98%, about 1% to about 99%, about 1% to about 100%, about 2% to about 5%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 95%, about 2% to about 98%, about 2% to about 99%, about 2% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 95%, about 5% to about 98%, about 5% to about 99%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 98%, about 10% to about 99%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 98%, about 20% to about 99%, about 20% to about 100%, about 30% to about 40%, about 30% to about 50%, about 30% to about 60%, about 30% to about 70%, about 30% to about 80%, about 30% to about 90%, about 30% to about 95%, about 30% to about 98%, about 30% to about 99%, about 30% to about 100%, about 40% to about 50%, about 40% to about 60%, about 40% to about 70%, about 40% to about 80%, about 40% to about 90%, about 40% to about 95%, about 40% to about 98%, about 40% to about 99%, about 40% to about 100%, about 50% to about 60%, about 50% to about 70%, about 50% to about 80%, about 50% to about 90%, about 50% to about 95%, about 50% to about 98%, about 50% to about 99%, about 50% to about 100%, about 60% to about 70%, about 60% to about 80%, about 60% to about 90%, about 60% to about 95%, about 60% to about 98%, about 60% to about 99%, about 60% to about 100%, about 70% to about 80%, about 70% to about 90%, about 70% to about 95%, about 70% to about 98%, about 70% to about 99%, about 70% to about 100%, about 80% to about 90%, about 80% to about 95%, about 80% to about 98%, about 80% to about 99%, about 80% to about 100%, about 90% to about 95%, about 90% to about 98%, about 90% to about 99%, about 90% to about 100%, about 95% to about 98%, about 95% to about 99%, about 95% to about 100%, about 98% to about 99%, about 98% to about 100%, or about 99% to about 100%.

Indoor cultivation may allow the CO₂ concentration level to be controlled during the growth of cannabis plants. An optimal CO₂ concentration level may be maintained over the entire plant life. An optimal CO₂ concentration level may be varied at different growth phases for a particular plant type. The optimal CO₂ concentration level may vary depending upon the species. The optimal CO₂ concentration level may be at least about 100 parts per million (ppm), 150 ppm, 200 ppm, 250 ppm, 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 950 ppm, 1000 ppm, 1050 ppm, 1100 ppm, 350 ppm, 1200 ppm, 1250 ppm, 1300 ppm, 1350 ppm, 1400 ppm, 1450 ppm, or at least about 1500 ppm. The optimal CO₂ concentration level may be no greater than about 1500 ppm, 1450 ppm, 1400 ppm, 1350 ppm, 1300 ppm, 1250 ppm, 1200 ppm, 350 ppm, 1100 ppm, 1050 ppm, 1000 ppm, 950 ppm, 900 ppm, 850 ppm, 800 ppm, 750 ppm, 700 ppm, 650 ppm, 600 ppm, 550 ppm, 500 ppm, 450 ppm, 400 ppm, 350 ppm, 300 ppm, 250 ppm, 200 ppm, 150 ppm, or no greater than about 100 ppm. The optimal CO₂ concentration level may occur in a range from about 100 ppm to about 200 ppm, about 100 ppm to about 300 ppm, about 100 ppm to about 400 ppm, about 100 ppm to about 500 ppm, about 100 ppm to about 600 ppm, about 100 ppm to about 700 ppm, about 100 ppm to about 800 ppm, about 100 ppm to about 900 ppm, about 100 ppm to about 1000 ppm, about 100 ppm to about 1100 ppm, about 100 ppm to about 1200 ppm, about 100 ppm to about 1300 ppm, about 100 ppm to about 1400 ppm, about 100 ppm to about 1500 ppm, about 200 ppm to about 300 ppm, about 200 ppm to about 400 ppm, about 200 ppm to about 500 ppm, about 200 ppm to about 600 ppm, about 200 ppm to about 700 ppm, about 200 ppm to about 800 ppm, about 200 ppm to about 900 ppm, about 200 ppm to about 1000 ppm, about 200 ppm to about 1100 ppm, about 200 ppm to about 1200 ppm, about 200 ppm to about 1300 ppm, about 200 ppm to about 1400 ppm, about 200 ppm to about 1500 ppm, about 300 ppm to about 400 ppm, about 300 ppm to about 500 ppm, about 300 ppm to about 600 ppm, about 300 ppm to about 700 ppm, about 300 ppm to about 800 ppm, about 300 ppm to about 900 ppm, about 300 ppm to about 1000 ppm, about 300 ppm to about 1100 ppm, about 300 ppm to about 1200 ppm, about 300 ppm to about 1300 ppm, about 300 ppm to about 1400 ppm, about 300 ppm to about 1500 ppm, about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1000 ppm, about 400 ppm to about 1100 ppm, about 400 ppm to about 1200 ppm, about 400 ppm to about 1300 ppm, about 400 ppm to about 1400 ppm, about 400 ppm to about 1500 ppm, about 500 ppm to about 600 ppm, about 500 ppm to about 700 ppm, about 500 ppm to about 800 ppm, about 500 ppm to about 900 ppm, about 500 ppm to about 1000 ppm, about 500 ppm to about 1100 ppm, about 500 ppm to about 1200 ppm, about 500 ppm to about 1300 ppm, about 500 ppm to about 1400 ppm, about 500 ppm to about 1500 ppm, about 600 ppm to about 700 ppm, about 600 ppm to about 800 ppm, about 600 ppm to about 900 ppm, about 600 ppm to about 1000 ppm, about 600 ppm to about 1100 ppm, about 600 ppm to about 1200 ppm, about 600 ppm to about 1300 ppm, about 600 ppm to about 1400 ppm, about 600 ppm to about 1500 ppm, about 700 ppm to about 800 ppm, about 700 ppm to about 900 ppm, about 700 ppm to about 1000 ppm, about 700 ppm to about 1100 ppm, about 700 ppm to about 1200 ppm, about 700 ppm to about 1300 ppm, about 700 ppm to about 1400 ppm, about 700 ppm to about 1500 ppm, about 800 ppm to about 900 ppm, about 800 ppm to about 1000 ppm, about 800 ppm to about 1100 ppm, about 800 ppm to about 1200 ppm, about 800 ppm to about 1300 ppm, about 800 ppm to about 1400 ppm, about 800 ppm to about 1500 ppm, about 900 ppm to about 1000 ppm, about 900 ppm to about 1100 ppm, about 900 ppm to about 1200 ppm, about 900 ppm to about 1300 ppm, about 900 ppm to about 1400 ppm, about 900 ppm to about 1500 ppm, about 1000 ppm to about 1100 ppm, about 1000 ppm to about 1200 ppm, about 1000 ppm to about 1300 ppm, about 1000 ppm to about 1400 ppm, about 1000 ppm to about 1500 ppm, about 1100 ppm to about 1200 ppm, about 1100 ppm to about 1300 ppm, about 1100 ppm to about 1400 ppm, about 1100 ppm to about 1500 ppm, about 1200 ppm to about 1300 ppm, about 1200 ppm to about 1400 ppm, about 1200 ppm to about 1500 ppm, about 1300 ppm to about 1400 ppm, about 1300 ppm to about 1500 ppm, or about 1400 ppm to about 1500 ppm.

Indoor cultivation may utilize light from within a defined region of the electromagnetic spectrum to enhance or optimize the growth of cannabis plants and alter other plant characteristics. An indoor cannabis cultivation process may involve one or more light sources with emission spectra within the ultraviolet, visible, or infrared wave bands. An indoor cannabis cultivation process may utilize multiple frequencies of light emission to enhance or optimize plant growth or alter other plant characteristics. Light frequencies may be selected based upon their affect upon photosynthesis or other photochemical reactions of relevance to plant biochemistry. Light frequencies may be less than about 1000 nanometers (nm), 900 nm, 800 nm, 700 nm, 600 nm, 500 nm, 400 nm, 300 nm, 200 nm, or no greater than about 100 nm. Light frequencies may greater than about 100 nm, 200 nm, 300 nm, 400 nm, 500 nm, 600 nm, 700 nm, 800 nm, 900 nm, or greater than about 1000 nm. Light frequencies may occur within a range from about 100 nm to about 200 nm, about 100 nm to about 300 nm, about 100 nm to about 400 nm, about 100 nm to about 500 nm, about 100 nm to about 600 nm, about 100 nm to about 700 nm, about 100 nm to about 800 nm, about 100 nm to about 900 nm, about 100 nm to about 1000 nm, about 200 nm to about 300 nm, about 200 nm to about 400 nm, about 200 nm to about 500 nm, about 200 nm to about 600 nm, about 200 nm to about 700 nm, about 200 nm to about 800 nm, about 200 nm to about 900 nm, about 200 nm to about 1000 nm, about 300 nm to about 400 nm, about 300 nm to about 500 nm, about 300 nm to about 600 nm, about 300 nm to about 700 nm, about 300 nm to about 800 nm, about 300 nm to about 900 nm, about 300 nm to about 1000 nm, about 400 nm to about 500 nm, about 400 nm to about 600 nm, about 400 nm to about 700 nm, about 400 nm to about 800 nm, about 400 nm to about 900 nm, about 400 nm to about 1000 nm, about 500 nm to about 600 nm, about 500 nm to about 700 nm, about 500 nm to about 800 nm, about 500 nm to about 900 nm, about 500 nm to about 1000 nm, about 600 nm to about 700 nm, about 600 nm to about 800 nm, about 600 nm to about 900 nm, about 600 nm to about 1000 nm, about 700 nm to about 800 nm, about 700 nm to about 900 nm, about 700 nm to about 1000 nm, about 800 nm to about 900 nm, about 800 nm to about 1000 nm, or about 900 nm to about 1000 nm.

Indoor cultivation may utilize light radiation of a defined intensity to enhance or optimize the growth of cannabis plants and alter other plant characteristics. An indoor cannabis cultivation process may involve one or more light sources with varying emission spectra. An indoor cannabis cultivation process may vary the intensity of emission for differing regions of the electromagnetic spectrum. For example, a lamp emitting radiation in the visible spectrum may radiate at a high intensity while an ultraviolet-emitting lamp may radiate at a lower intensity relative to the visible-spectrum lamp. Light sources and intensities may be configured to replicate sunlight. The average light intensity of an indoor cannabis cultivation process may be at least about 50 watts per square meter (W/m²), 100 W/m², 150 W/m², 200 W/m², 250 W/m², 300 W/m², 350 W/m², 400 W/m², 450 W/m², 500 W/m², 550 W/m², 600 W/m², 650 W/m², 700 W/m², 750 W/m², 800 W/m², 850 W/m², 900 W/m², 950 W/m², 1000 W/m², 1100 W/m², 1200 W/m², 1300 W/m², 1400 W/m², 1500 W/m², 1600 W/m², 1700 W/m², 1800 W/m², 1900 W/m², or at least about 2000 W/m². The average light intensity of an indoor cannabis cultivation process may be no greater than about 2000 W/m², 1900 W/m², 1800 W/m², 1700 W/m², 1600 W/m², 1500 W/m², 1400 W/m², 1300 W/m², 1200 W/m², 1100 W/m², 1000 W/m², 950 W/m², 900 W/m², 850 W/m², 800 W/m², 750 W/m², 700 W/m², 650 W/m², 600 W/m², 550 W/m², 500 W/m², 450 W/m², 400 W/m², 350 W/m², 300 W/m², 250 W/m², 200 W/m², 150 W/m², 100 W/m², or no greater that about 50 W/m². The average light intensity of an indoor cannabis cultivation process may occur in a range from about 50 W/m² to about 100 W/m², about 50 W/m² to about 300 W/m², about 50 W/m² to about 500 W/m², about 50 W/m² to about 700 W/m², about 50 W/m² to about 900 W/m², about 50 W/m² to about 1100 W/m², about 50 W/m² to about 1300 W/m², about 50 W/m² to about 1500 W/m², about 50 W/m² to about 1700 W/m², about 50 W/m² to about 1900 W/m², about 50 W/m² to about 2000 W/m², about 100 W/m² to about 300 W/m², about 100 W/m² to about 500 W/m², about 100 W/m² to about 700 W/m², about 100 W/m² to about 900 W/m², about 100 W/m² to about 1100 W/m², about 100 W/m² to about 1300 W/m², about 100 W/m² to about 1500 W/m², about 100 W/m² to about 1700 W/m², about 100 W/m² to about 1900 W/m², about 100 W/m² to about 2000 W/m², about 300 W/m² to about 500 W/m², about 300 W/m² to about 700 W/m², about 300 W/m² to about 900 W/m², about 300 W/m² to about 1100 W/m², about 300 W/m² to about 1300 W/m², about 300 W/m² to about 1500 W/m², about 300 W/m² to about 1700 W/m², about 300 W/m² to about 1900 W/m², about 300 W/m² to about 2000 W/m², about 500 W/m² to about 700 W/m², about 500 W/m² to about 900 W/m², about 500 W/m² to about 1100 W/m², about 500 W/m² to about 1300 W/m², about 500 W/m² to about 1500 W/m², about 500 W/m² to about 1700 W/m², about 500 W/m² to about 1900 W/m², about 500 W/m² to about 2000 W/m², about 700 W/m² to about 900 W/m², about 700 W/m² to about 1100 W/m², about 700 W/m² to about 1300 W/m², about 700 W/m² to about 1500 W/m², about 700 W/m² to about 1700 W/m², about 700 W/m² to about 1900 W/m², about 700 W/m² to about 2000 W/m², about 900 W/m² to about 1100 W/m², about 900 W/m² to about 1300 W/m², about 900 W/m² to about 1500 W/m², about 900 W/m² to about 1700 W/m², about 900 W/m² to about 1900 W/m², about 900 W/m² to about 2000 W/m², about 1100 W/m² to about 1300 W/m², about 1100 W/m² to about 1500 W/m², about 1100 W/m² to about 1700 W/m², about 1100 W/m² to about 1900 W/m², about 1100 W/m² to about 2000 W/m², about 1300 W/m² to about 1500 W/m², about 1300 W/m² to about 1700 W/m², about 1300 W/m² to about 1900 W/m², about 1300 W/m² to about 2000 W/m², about 1500 W/m² to about 1700 W/m², about 1500 W/m² to about 1900 W/m², about 1500 W/m² to about 2000 W/m², about 1700 W/m² to about 1900 W/m², about 1700 W/m² to about 2000 W/m², or about 1900 W/m² to about 2000 W/m².

Various methods may be utilized during growth and development of a Cannabis plant to affect one or more epigenetic modifications in the Cannabis plant. The one or more epigenetic modifications can induce a change in one or more properties (e.g., growth properties) of the Cannabis plant. The one or more properties can comprise cannabinoid yield, cannabinoid composition, terpene yield, terpene composition, bast fiber yield, plant vigor (e.g., rate of growth of the plant), dimensions (e.g., height, cross-sectional diameter, etc.), weight, color, odor, and/or other plant characteristics.

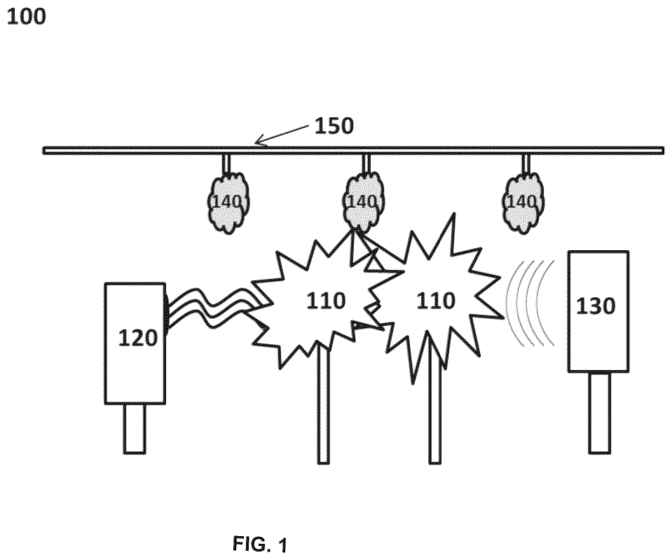

FIG. 1 depicts several methods for affecting cannabis development in a cannabis growing operation 100. Cannabis plants 110 may be subjected to one or more devices and/or compositions (e.g., liquid solutions, solid compositions, semi-solid compositions, gels, etc.) that are configured to induce one or more epigenetic modifications. In some cases, the one or more epigenetic modifications may regulate (e.g., accelerate or decelerate) an epigenetic drift in the Cannabis plants. The Cannabis plants 110 may be subjected to various devices and/or compositions that create the one or more epigenetic modifications from at least one abiotic stresses. For example, ultraviolet (UV) light sources or heat sources (e.g. infrared (IR) lamps) 120 may stress cannabis plants to create the one or more epigenetic modifications during foliage development. Alternatively or in addition to, acoustic sources 130 may use sound waves to create the one or more epigenetic modifications during foliage development of cannabis plants. Alternatively or in addition to, a misting system 150 may spray a solution 140 containing a cannabinoid precursor on plant foliage to increase cannabinoid yield.

The term “epigenetic drift” as used herein generally refers to the epigenetic modification occurring as a consequence of aging. The one or more epigenetic modifications induced by systems and methods provided herein can regulate an epigenetic drift in the Cannabis plants. In some cases, the one or more epigenetic modifications induced by systems and methods of the present disclosure may reduce or reverse the epigenetic drift in the Cannabis plants. In an example, the Cannabis plants may be subjected to an epigenetic drift in one or more genes operatively coupled to expression and/or biosynthesis of one or more cannabinoid compounds over time, resulting in a decreased expression and/or activity the gene. Examples of such genes may include, but are not limited to, CBD acid synthase (CBDAS), THC acid synthase (THCAS), and aromatic prenyltransferase (AP). Thus, the one or more epigenetic modifications induced by the systems and methods provided herein may reduce or reverse the epigenetic drift in the Cannabis plans, thereby maintaining or increasing the expression and/or biosynthesis of the one or more cannabinoid compounds. Alternatively or in addition to, the one or more epigenetic modifications induced by the systems and methods provided herein may initiate or accelerate the epigenetic drift in the Cannabis plants. In an example, the Cannabis plants may be subjected to an epigenetic drift in one or more genes (e.g., CBDAS, THCAS, AP, etc.) operatively coupled to expression and/or biosynthesis of one or more cannabinoid compounds over time, resulting in an increased expression and/or activity the gene. Thus, the one or more epigenetic modifications induced by the systems and methods provided herein may initiate or accelerate the epigenetic drift in the Cannabis plans, thereby increasing the expression and/or biosynthesis of the one or more cannabinoid compounds.

The epigenetic modifications in a Cannabis plant may occur in small molecules (e.g., hormones), genes (e.g., deoxyribonucleic acid (DNA) and/or ribonucleic acid (RNA)), and/or proteins (e.g., histones) of the Cannabis plant. The epigenetic modifications may comprise methylation, demethylation, phosphorylation, dephosphorylation, acetylation, deacetylation, ubiquitylation, deubiquitylation, sumoylation, desumoylation, ribosylation, deribosylation, citrullination (or deamination), and/or decitrullination. For example, the epigenetic modifications may induce demethylation in one or more genes (e.g., CBDAS, THCAS, AP, etc.) operatively coupled to expression and/or biosynthesis of one or more cannabinoid compounds.

The epigenetic modifications can be stable. In some cases, the epigenetic modifications can be stable (or can persist) for at least about 10 minutes, 30 minutes, 1 hour, 2 hours, 4 hours, 6 hours, 8 hours, 12 hours, 18 hours, 1 day, 2 days, 3 days, 4 days, 5 days, 6 days, 7 days, 10 days, 14 days, 21 days, 28 days, 1 month, 2 months, 3 months, 4 months, 5 months, 6 months, 9 months, 1 year, 2 years, 5 years, or more after the occurrence of the epigenetic modifications. Alternatively, the epigenetic modifications can be permanent. In another alternative, the epigenetic modifications can be temporary. In some cases, the epigenetic modifications can be reversed (e.g., from methylation to demethylation, from demethylation to methylation, from acetylation to deacetylation, from deacetylation to acetylation, etc.) following at most about 5 years, 2 years, 1 year, 9 months, 6 months, 5 months, 4 months, 3 months, 2 months, 1 month, 28 days, 21 days, 14 days, 7 days, 6 days, 5 days, 4 days, 3 days, 2 days, 1 day, 18 hours, 12 hours, 8 hours, 6 hours, 4 hours, 2 hours, 1 hour, 30 minutes, 10 minutes, or less of the occurrence of the epigenetic modifications.

The epigenetic modifications in the Cannabis plants may be heritable to a progeny of the Cannabis plants. Alternatively, the epigenetic modifications in the Cannabis plants may not be heritable to the progeny.

The epigenetic modification in the Cannabis plants may induce an increased level of cannabinoid (e.g., CBD, THC, etc.) expression in the Cannabis plants. The increased level of cannabinoid expression may be at least at least about 1%, 5%, 10%, 20%, 30%, 40%, 50%, 75%, 100%, 150%, 200%, 300%, 400%, 500%, or more in comparison to Cannabis plants without the epigenetic modification. The increased level of cannabinoid expression may be at most about 500%, 400%, 300%, 200%, 150%, 100%, 75%, 50%, 40%, 30%, 20%, 10%, 5%, 1%, or less in comparison to Cannabis plants without the epigenetic modification. Alternatively, the epigenetic modification in the Cannabis plants may induce a decreased level of cannabinoid (e.g., CBD, THC, etc.) expression in the Cannabis plants. The decreased level of cannabinoid expression may be at least at least about 1%, 5%, 10%, 20%, 30%, 40%, 50%, 75%, 100%, 150%, 200%, 300%, 400%, 500%, or more in comparison to Cannabis plants without the epigenetic modification. The decreased level of cannabinoid expression may be at most about 500%, 400%, 300%, 200%, 150%, 100%, 75%, 50%, 40%, 30%, 20%, 10%, 5%, 1%, or less in comparison to Cannabis plants without the epigenetic modification

The epigenetic modification in the Cannabis plants may induce an increased level of terpene expression in the Cannabis plants. The increased level of terpene expression may be at least at least about 1%, 5%, 10%, 20%, 30%, 40%, 50%, 75%, 100%, 150%, 200%, 300%, 400%, 500%, or more in comparison to Cannabis plants without the epigenetic modification. The increased level of terpene expression may be at most about 500%, 400%, 300%, 200%, 150%, 100%, 75%, 50%, 40%, 30%, 20%, 10%, 5%, 1%, or less in comparison to Cannabis plants without the epigenetic modification. Alternatively, the epigenetic modification in the Cannabis plants may induce a decreased level of terpene expression in the Cannabis plants. The decreased level of terpene expression may be at least at least about 1%, 5%, 10%, 20%, 30%, 40%, 50%, 75%, 100%, 150%, 200%, 300%, 400%, 500%, or more in comparison to Cannabis plants without the epigenetic modification. The decreased level of terpene expression may be at most about 500%, 400%, 300%, 200%, 150%, 100%, 75%, 50%, 40%, 30%, 20%, 10%, 5%, 1%, or less in comparison to Cannabis plants without the epigenetic modification

The epigenetic modification in the Cannabis plants may induce a change in the composition of expressed compounds (e.g., cannabinoids, terpenes, etc.) in the Cannabis plants. For example, the epigenetic modification may change an amount of terpene relative to an amount of cannabinoids in the Cannabis plants. The relative amount of terpene relative to the amount of cannabinoids may increase at least about 1%, 5%, 10%, 20%, 30%, 40%, 50%, 75%, 100%, 150%, 200%, 300%, 400%, 500%, or more. The relative amount of terpene relative to the amount of cannabinoids may increase at most about 500%, 400%, 300%, 200%, 150%, 100%, 75%, 50%, 40%, 30%, 20%, 10%, 5%, 1%, or less. Alternatively, the relative amount of terpene relative to the amount of cannabinoids may decrease at least about 1%, 5%, 10%, 20%, 30%, 40%, 50%, 75%, 100%, 150%, 200%, 300%, 400%, 500%, or more. The relative amount of terpene relative to the amount of cannabinoids may decrease at most about 500%, 400%, 300%, 200%, 150%, 100%, 75%, 50%, 40%, 30%, 20%, 10%, 5%, 1%, or less. In another example, the epigenetic modification may change an amount of CBD relative to an amount of THC in the Cannabis plants. The relative amount of CBD relative to the amount of THC may increase at least about 1%, 5%, 10%, 20%, 30%, 40%, 50%, 75%, 100%, 150%, 200%, 300%, 400%, 500%, or more. The relative amount of CBD relative to the amount of THC may increase at most about 500%, 400%, 300%, 200%, 150%, 100%, 75%, 50%, 40%, 30%, 20%, 10%, 5%, 1%, or less. Alternatively, the relative amount of CBD relative to the amount of THC may decrease at least about 1%, 5%, 10%, 20%, 30%, 40%, 50%, 75%, 100%, 150%, 200%, 300%, 400%, 500%, or more. The relative amount of CBD relative to the amount of THC may decrease at most about 500%, 400%, 300%, 200%, 150%, 100%, 75%, 50%, 40%, 30%, 20%, 10%, 5%, 1%, or less. The epigenetic modification in the Cannabis plants may induce a change in growth of the Cannabis plants. The epigenetic modification may increase a rate of growth of the Cannabis plants by at least about 1%, 5%, 10%, 20%, 30%, 40%, 50%, 75%, 100%, 150%, 200%, 300%, 400%, 500%, or more in comparison to Cannabis plants without the epigenetic modifications. The epigenetic modification may increase the rate of growth of the Cannabis plants by at most about 500%, 400%, 300%, 200%, 150%, 100%, 75%, 50%, 40%, 30%, 20%, 10%, 5%, 1%, or less in comparison to Cannabis plants without the epigenetic modifications. Alternatively, the epigenetic modification may decrease a rate of growth of the Cannabis plants by at least about 1%, 5%, 10%, 20%, 30%, 40%, 50%, 75%, 100%, 150%, 200%, 300%, 400%, 500%, or more in comparison to Cannabis plants without the epigenetic modifications. The epigenetic modification may decrease the rate of growth of the Cannabis plants by at most about 500%, 400%, 300%, 200%, 150%, 100%, 75%, 50%, 40%, 30%, 20%, 10%, 5%, 1%, or less in comparison to Cannabis plants without the epigenetic modifications.

Two or more different types of treatment may be administered in sequence and/or at different points in time. In some instances, two or more different types of treatment may be administered simultaneously. For example, a treatment regimen may involve administration of the following treatments in the following sequence: UV exposure, then acoustic exposure, then solution exposure; UV exposure, then solution exposure, then acoustic exposure; acoustic exposure, then UV exposure, then solution exposure; acoustic exposure, then solution exposure, then UV exposure; solution exposure, then UV exposure, then acoustic exposure; solution exposure then acoustic exposure, then UV exposure; UV exposure, then simultaneous exposure of acoustic and solution; acoustic exposure, then simultaneous exposure of UV and solution; solution exposure, then simultaneous exposure of UV and acoustic; simultaneous exposure of UV and acoustic, then solution exposure; simultaneous exposure of UV and solution, then acoustic exposure; simultaneous exposure of acoustic and solution, then UV exposure; simultaneous exposure of UV, acoustic, and solution; only UV exposure; only acoustic exposure; or only solution exposure. In some instances, a first type of treatment (e.g., solution treatment) may facilitate or interrupt reception of a second type of treatment (e.g., UV treatment, acoustic treatment).

Such treatments, such as UV treatment, acoustic treatment, temperature exposure, and/or chemical exposure, may be administered in an indoor environment. Alternatively, such treatments may be administered in an outdoor environment, such as in an ambient environment. In some instances, plants may be transferred from a first environment to a second environment, and optionally to an Nth environment, prior to, during, or subsequent to a treatment, where the first and second environments are different types of environments. For example, the first environment can be an indoor environment and the second environment can be an outdoor environment, or vice versa. In another example, the first environment can be a first controlled environment (e.g., having a first humidity, first temperature, first pressure, etc.) and the second environment can be a second controlled environment (e.g., having a second humidity, second temperature, second pressure, etc.). In some instances, a plant may be subject to a first type of treatment (e.g., UV exposure) in a first environment, and a second type of treatment (e.g., acoustic exposure) in a second environment, and a third type of treatment (e.g., chemical exposure) in a third environment. In other instances, two or more different types of treatment may be administered in the same environment.

Exposure to UV light may be utilized to promote one or more epigenetic modifications in cannabis plants by causing an abiotic stress. Exposure to UV light may provoke genomic, proteomic, or transcriptomic changes to the cannabis plant that may alter one or more properties of the plant during its development. UV light may alter the cannabinoid expression profiles of the cannabis plant. UV light may alter the yield of cannabinoids from the cannabis plant. UV light may enhance the vigor of plant growth. UV light may cause photochemical reactions that result in cannabinoid shifts, such as converting THC to cannabinol (CBN). Exposure to UV light may occur during different stages of plant growth and development, including the seedling, vegetative, budding, flowering, and ripening stages. UV light exposure may occur during the development of true-type foliage. UV light exposure may occur periodically or continuously at varying intensity levels. UV light exposure may occur over a broad spectrum of UV wavelengths or may be narrowed to a particular effective frequency. UV light intensity may be low, moderate, or high during treatment of the cannabis plants. UV light exposure may be administered in a single occurrence or in multiple doses (e.g., multiple distinct exposure events). UV light exposure may be administered at regular or irregular intervals.

UV light exposure may occur continuously, or at a frequency. For example, UV light exposure may occur at a frequency of about every hour, every 2 hours, every 4 hours, every 6 hours, every 8 hours, every 12 hours, every 24 hours, every 2 days, every 3 days, every week, or about every other week. UV light exposure may occur at a frequency of at least about every hour, every 2 hours, every 4 hours, every 6 hours, every 8 hours, every 12 hours, every 24 hours, every 2 days, every 3 days, every week, or at least about every other week. UV light exposure may occur at a frequency of no more than about every other week, every week, every 3 days, every 2 days, every 24 hours, every 12 hours, every 8 hours, every 6 hours, every 4 hours, every 3 hours, every 2 hours, or no more than about every hour. UV light treatments may be administered for a period of about 1 day, 2 days, 3 days, 4 days, 5 days, 6 days, 7 days, 10 days, 14 days, 21 days, 28 days, 1 month, 2 months, 3 months, 4 months, 5 months, or about 6 months. UV light treatments may be administered for a period of at least about 1 day, 2 days, 3 days, 4 days, 5 days, 6 days, 7 days, 10 days, 14 days, 21 days, 28 days, 1 month, 2 months, 3 months, 4 months, 5 months, or about 6 months. UV light exposure may be administered for a period of no more than about 6 months, 5 months, 4 months, 3 months, 2 months, 1 month, 28 days, 21 days, 14 days, 10 days, 7 days, 6 days, 5 days, 4 days, 3 days, 2 days, or no more than about 1 day. UV light exposure may be administered for a period in a range from about 1 day to about 7 days, about 1 day to about 14 days, about 1 day to about 21 days, about 1 day to about 28 days, about 1 day to about 1 month, about 1 day to about 3 months, about 1 day to about 6 months, about 7 days to about 14 days, about 7 days to about 21 days, about 7 days to about 28 days, about 7 days to about 1 month, about 7 days to about 3 months, about 7 days to about 6 months, about 14 days to about 21 days, about 14 days to about 28 days, about 14 days to about 1 month, about 14 days to about 3 months, about 14 days to about 6 months, about 21 days to about 28 days, about 21 days to about 1 month, about 21 days to about 3 months, about 21 days to about 6 months, about 28 days to about 1 month, about 28 days to about 3 months, about 28 days to about 6 months, about 1 month to about 3 months, about 1 month to about 6 months, or about 3 months to about 6 months.

A UV light treatment may use UV light having a wavelength from between about 10 nanometers to about 400 nanometers. The UV light used in the UV light treatment may have a wavelength of at least about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm or more. Alternatively or in addition, the UV light used in the UV light treatment may have a wavelength of at most about 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm, 210 nm, 200 nm, 190 nm, 180 nm, 170 nm, 160 nm, 150 nm, 140 nm, 130 nm, 120 nm, 110 nm, 100 nm, 90 nm, 80 nm, 70 nm, 60 nm, 50 nm, 40 nm, 30 nm, 20 nm, 10 nm or less. In some instances, alternative to or in addition to UV light, non-UV range electromagnetic radiation may be used in a similar manner, for example, including light in the gamma ray range, x-ray range, visible range, infrared range, microwave range, or radio range.

Acoustic exposure may be utilized to promote one or more epigenetic modifications in cannabis plants. Acoustic exposure may provoke genomic, proteomic, or transcriptomic changes to the cannabis plant that may alter one or more properties of the plant during its development. Acoustic exposure may alter the cannabinoid expression profiles of the cannabis plant. Acoustic exposure may alter the yield of cannabinoids from the cannabis plant. Acoustic exposure may enhance the vigor of plant growth. Acoustic exposure may occur during different stages of plant growth and development, including the seeding, vegetative, budding, flowering, and ripening stages. Acoustic exposure may occur during the development of true-type foliage. Acoustic exposure may occur periodically or continuously at varying intensity levels. Acoustic exposure may occur over a broad spectrum of frequencies or may be narrowed to a particular effective frequency. Acoustic exposure intensity may be low, moderate, or high during treatment of the cannabis plants. Acoustic exposure may be administered in a single occurrence or in multiple doses (e.g., multiple distinct exposure events). Acoustic light exposure may be administered at regular or irregular intervals.

Acoustic exposure may occur continuously, or at a frequency. For example, acoustic exposure may occur at a frequency of about every hour, every 2 hours, every 4 hours, every 6 hours, every 8 hours, every 12 hours, every 24 hours, every 2 days, every 3 days, every week, or about every other week. Acoustic exposure may occur at a frequency of at least about every hour, every 2 hours, every 4 hours, every 6 hours, every 8 hours, every 12 hours, every 24 hours, every 2 days, every 3 days, every week, or at least about every other week. Acoustic exposure may occur at a frequency of no more than about every other week, every week, every 3 days, every 2 days, every 24 hours, every 12 hours, every 8 hours, every 6 hours, every 4 hours, every 3 hours, every 2 hours, or no more than about every hour. Acoustic exposure treatments may be administered for a period of about 1 day, 2 days, 3 days, 4 days, 5 days, 6 days, 7 days, 10 days, 14 days, 21 days, 28 days, 1 month, 2 months, 3 months, 4 months, 5 months, or about 6 months. Acoustic exposure treatments may be administered for a period of at least about 1 day, 2 days, 3 days, 4 days, 5 days, 6 days, 7 days, 10 days, 14 days, 21 days, 28 days, 1 month, 2 months, 3 months, 4 months, 5 months, or about 6 months. Acoustic exposure may be administered for a period of no more than about 6 months, 5 months, 4 months, 3 months, 2 months, 1 month, 28 days, 21 days, 14 days, 10 days, 7 days, 6 days, 5 days, 4 days, 3 days, 2 days, or no more than about 1 day Acoustic exposure may be administered for a period in a range from about 1 day to about 7 days, about 1 day to about 14 days, about 1 day to about 21 days, about 1 day to about 28 days, about 1 day to about 1 month, about 1 day to about 3 months, about 1 day to about 6 months, about 7 days to about 14 days, about 7 days to about 21 days, about 7 days to about 28 days, about 7 days to about 1 month, about 7 days to about 3 months, about 7 days to about 6 months, about 14 days to about 21 days, about 14 days to about 28 days, about 14 days to about 1 month, about 14 days to about 3 months, about 14 days to about 6 months, about 21 days to about 28 days, about 21 days to about 1 month, about 21 days to about 3 months, about 21 days to about 6 months, about 28 days to about 1 month, about 28 days to about 3 months, about 28 days to about 6 months, about 1 month to about 3 months, about 1 month to about 6 months, or about 3 months to about 6 months.

Acoustic exposure for cannabis plants may be administered at a chosen frequency and a chosen intensity. The acoustic frequency may be about 1 hertz (Hz), 5 Hz, 10 Hz, 15 Hz, 20 Hz, 25 Hz, 30 Hz, 35 Hz, 40 Hz, 45 Hz, 50 Hz, 55 Hz, 60 Hz, 65 Hz, 70 Hz, 75 Hz, 80 Hz, 85 Hz, 90 Hz, 100 Hz, 110 Hz, 120 Hz, 130 Hz, 140 Hz, 150 Hz, 160 Hz, 170 Hz, 180 Hz, 190 Hz, 200 Hz, 210 Hz, 220 Hz, 230 Hz, 240 Hz, 250 Hz, 260 Hz, 270 Hz, 280 Hz, 290 Hz, 300 Hz, 350 Hz, 400 Hz, 450 Hz, 500 Hz, 550 Hz, 600 Hz, 650 Hz, 700 Hz, 750 Hz, 800 Hz, 850 Hz, 900 Hz, 950 Hz, 1000 Hz, 1100 Hz, 1200 Hz, 1300 Hz, 1400 Hz, 1500 Hz, 1600 Hz, 1700 Hz, 1800 Hz, 1900 Hz, 2000 Hz, 2100 Hz, 2200 Hz, 2300 Hz, 2400 Hz, 2500 Hz, 2600 Hz, 2700 Hz, 2800 Hz, 2900 Hz, 3000 Hz, 3100 Hz, 3200 Hz, 3300 Hz, 3400 Hz, 3500 Hz, 3600 Hz, 3700 Hz, 3800 Hz, 3900 Hz, 4000 Hz, 4100 Hz, 4200 Hz, 4300 Hz, 4400 Hz, 4500 Hz, 4600 Hz, 4700 Hz, 4800 Hz, 4900 Hz, or about 5000 Hz. The acoustic frequency may be no less than about 1 Hz, 5 Hz, 10 Hz, 15 Hz, 20 Hz, 25 Hz, 30 Hz, 35 Hz, 40 Hz, 45 Hz, 50 Hz, 55 Hz, 60 Hz, 65 Hz, 70 Hz, 75 Hz, 80 Hz, 85 Hz, 90 Hz, 100 Hz, 110 Hz, 120 Hz, 130 Hz, 140 Hz, 150 Hz, 160 Hz, 170 Hz, 180 Hz, 190 Hz, 200 Hz, 210 Hz, 220 Hz, 230 Hz, 240 Hz, 250 Hz, 260 Hz, 270 Hz, 280 Hz, 290 Hz, 300 Hz, 350 Hz, 400 Hz, 450 Hz, 500 Hz, 550 Hz, 600 Hz, 650 Hz, 700 Hz, 750 Hz, 800 Hz, 850 Hz, 900 Hz, 950 Hz, 1000 Hz, 1100 Hz, 1200 Hz, 1300 Hz, 1400 Hz, 1500 Hz, 1600 Hz, 1700 Hz, 1800 Hz, 1900 Hz, 2000 Hz, 2100 Hz, 2200 Hz, 2300 Hz, 2400 Hz, 2500 Hz, 2600 Hz, 2700 Hz, 2800 Hz, 2900 Hz, 3000 Hz, 3100 Hz, 3200 Hz, 3300 Hz, 3400 Hz, 3500 Hz, 3600 Hz, 3700 Hz, 3800 Hz, 3900 Hz, 4000 Hz, 4100 Hz, 4200 Hz, 4300 Hz, 4400 Hz, 4500 Hz, 4600 Hz, 4700 Hz, 4800 Hz, 4900 Hz, or no less than about 5000 Hz. The acoustic frequency may be no greater than about 5000 Hz, 4900 Hz, 4800 Hz, 4700 Hz, 4600 Hz, 4500 Hz, 4400 Hz, 4300 Hz, 4200 Hz, 4100 Hz, 4000 Hz, 3900 Hz, 3800 Hz, 3700 Hz, 3600 Hz, 3500 Hz, 3400 Hz, 3300 Hz, 3200 Hz, 3100 Hz, 3000 Hz, 2900 Hz, 2800 Hz, 2700 Hz, 2600 Hz, 2500 Hz, 2400 Hz, 2300 Hz, 2200 Hz, 2100 Hz, 2000 Hz, 1900 Hz, 1800 Hz, 1700 Hz, 1600 Hz, 1500 Hz, 1400 Hz, 1300 Hz, 1200 Hz, 1100 Hz, 1000 Hz, 950 Hz, 900 Hz, 850 Hz, 800 Hz, 750 Hz, 700 Hz, 650 Hz, 600 Hz, 550 Hz, 500 Hz, 450 Hz, 400 Hz, 350 Hz, 300 Hz, 290 Hz, 280 Hz, 270 Hz, 260 Hz, 250 Hz, 240 Hz, 230 Hz, 220 Hz, 210 Hz, 200 Hz, 190 Hz, 180 Hz, 170 Hz, 160 Hz, 150 Hz, 140 Hz, 130 Hz, 120 Hz, 110 Hz, 100 Hz, 90 Hz, 85 Hz, 80 Hz, 75 Hz, 70 Hz, 65 Hz, 60 Hz, 55 Hz, 50 Hz, 45 Hz, 40 Hz, 35 Hz, 30 Hz, 25 Hz, 20 Hz, 15 Hz, 10 Hz, 5 Hz, or no greater than about 1 Hz. The acoustic frequency may occur in a range from about 1 Hz to about 20 Hz, about 1 Hz to about 50 Hz, about 1 Hz to about 100 Hz, about 1 Hz to about 200 Hz, about 1 Hz to about 300 Hz, about 1 Hz to about 400 Hz, about 1 Hz to about 500 Hz, about 1 Hz to about 600 Hz, about 1 Hz to about 700 Hz, about 1 Hz to about 800 Hz, about 1 Hz to about 900 Hz, about 1 Hz to about 1000 Hz, about 1 Hz to about 1500 Hz, about 1 Hz to about 2000 Hz, about 1 Hz to about 2500 Hz, about 1 Hz to about 3000 Hz, about 1 Hz to about 3500 Hz, about 1 Hz to about 4000 Hz, about 1 Hz to about 4500 Hz, about 1 Hz to about 5000 Hz, about 20 Hz to about 50 Hz, about 20 Hz to about 100 Hz, about 20 Hz to about 200 Hz, about 20 Hz to about 300 Hz, about 20 Hz to about 400 Hz, about 20 Hz to about 500 Hz, about 20 Hz to about 600 Hz, about 20 Hz to about 700 Hz, about 20 Hz to about 800 Hz, about 20 Hz to about 900 Hz, about 20 Hz to about 1000 Hz, about 20 Hz to about 1500 Hz, about 20 Hz to about 2000 Hz, about 20 Hz to about 2500 Hz, about 20 Hz to about 3000 Hz, about 20 Hz to about 3500 Hz, about 20 Hz to about 4000 Hz, about 20 Hz to about 4500 Hz, about 20 Hz to about 5000 Hz, about 50 Hz to about 100 Hz, about 50 Hz to about 200 Hz, about 50 Hz to about 300 Hz, about 50 Hz to about 400 Hz, about 50 Hz to about 500 Hz, about 50 Hz to about 600 Hz, about 50 Hz to about 700 Hz, about 50 Hz to about 800 Hz, about 50 Hz to about 900 Hz, about 50 Hz to about 1000 Hz, about 50 Hz to about 1500 Hz, about 50 Hz to about 2000 Hz, about 50 Hz to about 2500 Hz, about 50 Hz to about 3000 Hz, about 50 Hz to about 3500 Hz, about 50 Hz to about 4000 Hz, about 50 Hz to about 4500 Hz, about 50 Hz to about 5000 Hz, about 100 Hz to about 200 Hz, about 100 Hz to about 300 Hz, about 100 Hz to about 400 Hz, about 100 Hz to about 500 Hz, about 100 Hz to about 600 Hz, about 100 Hz to about 700 Hz, about 100 Hz to about 800 Hz, about 100 Hz to about 900 Hz, about 100 Hz to about 1000 Hz, about 100 Hz to about 1500 Hz, about 100 Hz to about 2000 Hz, about 100 Hz to about 2500 Hz, about 100 Hz to about 3000 Hz, about 100 Hz to about 3500 Hz, about 100 Hz to about 4000 Hz, about 100 Hz to about 4500 Hz, about 100 Hz to about 5000 Hz, about 200 Hz to about 300 Hz, about 200 Hz to about 400 Hz, about 200 Hz to about 500 Hz, about 200 Hz to about 600 Hz, about 200 Hz to about 700 Hz, about 200 Hz to about 800 Hz, about 200 Hz to about 900 Hz, about 200 Hz to about 1000 Hz, about 200 Hz to about 200500 Hz, about 200 Hz to about 2000 Hz, about 200 Hz to about 2500 Hz, about 200 Hz to about 3000 Hz, about 200 Hz to about 3500 Hz, about 200 Hz to about 4000 Hz, about 200 Hz to about 4500 Hz, about 200 Hz to about 5000 Hz, about 300 Hz to about 400 Hz, about 300 Hz to about 500 Hz, about 300 Hz to about 600 Hz, about 300 Hz to about 700 Hz, about 300 Hz to about 800 Hz, about 300 Hz to about 900 Hz, about 300 Hz to about 1000 Hz, about 300 Hz to about 1500 Hz, about 300 Hz to about 2000 Hz, about 300 Hz to about 2500 Hz, about 300 Hz to about 3000 Hz, about 300 Hz to about 3500 Hz, about 300 Hz to about 4000 Hz, about 300 Hz to about 4500 Hz, about 300 Hz to about 5000 Hz, about 400 Hz to about 500 Hz, about 400 Hz to about 600 Hz, about 400 Hz to about 700 Hz, about 400 Hz to about 800 Hz, about 400 Hz to about 900 Hz, about 400 Hz to about 1000 Hz, about 400 Hz to about 1500 Hz, about 400 Hz to about 2000 Hz, about 400 Hz to about 2500 Hz, about 400 Hz to about 3000 Hz, about 400 Hz to about 3500 Hz, about 400 Hz to about 4000 Hz, about 400 Hz to about 4500 Hz, about 400 Hz to about 5000 Hz, about 500 Hz to about 600 Hz, about 500 Hz to about 700 Hz, about 500 Hz to about 800 Hz, about 500 Hz to about 900 Hz, about 500 Hz to about 1000 Hz, about 500 Hz to about 1500 Hz, about 500 Hz to about 2000 Hz, about 500 Hz to about 2500 Hz, about 500 Hz to about 3000 Hz, about 500 Hz to about 3500 Hz, about 500 Hz to about 4000 Hz, about 500 Hz to about 4500 Hz, about 500 Hz to about 5000 Hz, about 600 Hz to about 700 Hz, about 600 Hz to about 800 Hz, about 600 Hz to about 900 Hz, about 600 Hz to about 1000 Hz, about 600 Hz to about 1500 Hz, about 600 Hz to about 2000 Hz, about 600 Hz to about 2500 Hz, about 600 Hz to about 3000 Hz, about 600 Hz to about 3500 Hz, about 600 Hz to about 4000 Hz, about 600 Hz to about 4500 Hz, about 600 Hz to about 5000 Hz, about 700 Hz to about 800 Hz, about 700 Hz to about 900 Hz, about 700 Hz to about 1000 Hz, about 700 Hz to about 1500 Hz, about 700 Hz to about 2000 Hz, about 700 Hz to about 2500 Hz, about 700 Hz to about 3000 Hz, about 700 Hz to about 3500 Hz, about 700 Hz to about 4000 Hz, about 700 Hz to about 4500 Hz, about 700 Hz to about 5000 Hz, about 800 Hz to about 900 Hz, about 800 Hz to about 1000 Hz, about 800 Hz to about 1500 Hz, about 800 Hz to about 2000 Hz, about 800 Hz to about 2500 Hz, about 800 Hz to about 3000 Hz, about 800 Hz to about 3500 Hz, about 800 Hz to about 4000 Hz, about 800 Hz to about 4500 Hz, about 800 Hz to about 5000 Hz, about 900 Hz to about 1000 Hz, about 900 Hz to about 1500 Hz, about 900 Hz to about 2000 Hz, about 900 Hz to about 2500 Hz, about 900 Hz to about 3000 Hz, about 900 Hz to about 3500 Hz, about 900 Hz to about 4000 Hz, about 900 Hz to about 4500 Hz, about 900 Hz to about 5000 Hz, about 1000 Hz to about 1500 Hz, about 1000 Hz to about 2000 Hz, about 1000 Hz to about 2500 Hz, about 1000 Hz to about 3000 Hz, about 1000 Hz to about 3500 Hz, about 1000 Hz to about 4000 Hz, about 1000 Hz to about 4500 Hz, about 1000 Hz to about 5000 Hz, about 1500 Hz to about 2000 Hz, about 1500 Hz to about 2500 Hz, about 1500 Hz to about 3000 Hz, about 1500 Hz to about 3500 Hz, about 1500 Hz to about 4000 Hz, about 1500 Hz to about 4500 Hz, about 1500 Hz to about 5000 Hz, about 2000 Hz to about 2500 Hz, about 2000 Hz to about 3000 Hz, about 2000 Hz to about 3500 Hz, about 2000 Hz to about 4000 Hz, about 2000 Hz to about 4500 Hz, about 2000 Hz to about 5000 Hz, about 2500 Hz to about 3000 Hz, about 2500 Hz to about 3500 Hz, about 2500 Hz to about 4000 Hz, about 2500 Hz to about 4500 Hz, about 2500 Hz to about 5000 Hz, about 3000 Hz to about 3500 Hz, about 3000 Hz to about 4000 Hz, about 3000 Hz to about 4500 Hz, about 3000 Hz to about 5000 Hz, about 3500 Hz to about 4000 Hz, about 3500 Hz to about 4500 Hz, about 3500 Hz to about 5000 Hz, about 4000 Hz to about 4500 Hz, about 4000 Hz to about 5000 Hz, or about 4500 Hz to about 5000 Hz.

The intensity of acoustic exposure to cannabis plants may be about 1 decibel (dB), 5 dB, 10 dB, 15 dB, 20 dB, 25 dB, 30 dB, 35 dB, 40 dB, 45 dB, 50 dB, 55 dB, 60 dB, 65 dB, 70 dB, 75 dB, 80 dB, 85 dB, 90 dB, 95 dB, 100 dB, 105 dB, 110 dB, 115 dB, 120 dB, 125 dB, 130 dB, 135 dB, 140 dB, 145 dB, 150 dB, 155 dB, 160 dB, 165 dB, 170 dB, 175 dB, or about 180 dB. The intensity of acoustic exposure may be no less than about 1 dB, 5 dB, 10 dB, 15 dB, 20 dB, 25 dB, 30 dB, 35 dB, 40 dB, 45 dB, 50 dB, 55 dB, 60 dB, 65 dB, 70 dB, 75 dB, 80 dB, 85 dB, 90 dB, 95 dB, 100 dB, 105 dB, 110 dB, 115 dB, 120 dB, 125 dB, 130 dB, 135 dB, 140 dB, 145 dB, 150 dB, 155 dB, 160 dB, 165 dB, 170 dB, 175 dB, or about 180 dB. The acoustic exposure intensity may be no greater than about 180 dB, 175 dB, 170 dB, 165 dB, 160 dB, 155 dB, 150 dB, 145 dB, 140 dB, 135 dB, 130 dB, 125 dB, 120 dB, 115 dB, 110 dB, 105 dB, 100 dB, 95 dB, 90 dB, 85 dB, 80 dB, 75 dB, 70 dB, 65 dB, 60 dB, 55 dB, 50 dB, 45 dB, 40 dB, 35 dB, 30 dB, 25 dB, 20 dB, 15 dB, 10 dB, 5 dB, or no greater than about 1 dB. The acoustic exposure intensity for cannabis plants may occur in a range from about 1 dB to about 20 dB, about 1 dB to about 40 dB, about 1 dB to about 60 dB, about 1 dB to about 80 dB, about 1 dB to about 100 dB, about 1 dB to about 120 dB, about 1 dB to about 140 dB, about 1 dB to about 160 dB, about 1 dB to about 180 dB, about 20 dB to about 40 dB, about 20 dB to about 60 dB, about 20 dB to about 80 dB, about 20 dB to about 100 dB, about 20 dB to about 120 dB, about 20 dB to about 140 dB, about 20 dB to about 160 dB, about 20 dB to about 180 dB, about 40 dB to about 60 dB, about 40 dB to about 80 dB, about 40 dB to about 100 dB, about 40 dB to about 120 dB, about 40 dB to about 140 dB, about 40 dB to about 160 dB, about 40 dB to about 180 dB, about 60 dB to about 80 dB, about 60 dB to about 100 dB, about 60 dB to about 120 dB, about 60 dB to about 140 dB, about 60 dB to about 160 dB, about 60 dB to about 180 dB, about 80 dB to about 100 dB, about 80 dB to about 120 dB, about 80 dB to about 140 dB, about 80 dB to about 160 dB, about 80 dB to about 180 dB, about 100 dB to about 120 dB, about 100 dB to about 140 dB, about 100 dB to about 160 dB, about 100 dB to about 180 dB, about 120 dB to about 140 dB, about 120 dB to about 160 dB, about 120 dB to about 180 dB, about 140 dB to about 160 dB, about 140 dB to about 180 dB, or about 160 dB to about 180 dB.

Temperature exposure may be utilized to generate one or more epigenetic modifications in cannabis plants. Temperature exposure may occur may include heat exposure and cold exposure as methods of creating an abiotic stress in the treated cannabis plants. Methods of creating heat exposure may include infrared (IR) lamps, electrical heaters, and burners. Methods of creating cold exposure may include refrigeration methods and icing of cannabis plants. Temperature exposure methods may involve altering the environmental temperature of a cannabis plant by a certain amount relative to an optimal growth temperature for a given amount of time. A temperature exposure method may involve heating or cooling by about 5° C., 10° C., 15° C., 20° C., 25° C., 30° C., 35° C., 40° C., 45° C., or about 50° C. A temperature exposure method may involve heating or cooling by at least about 5° C., 10° C., 15° C., 20° C., 25° C., 30° C., 35° C., 40° C., 45° C., or about 50° C. or more. A temperature exposure method may involve heating or cooling by no more than about 50° C., 45° C., 40° C., 35° C., 30° C., 25° C., 20° C., 15° C., 10° C., or no more than about 5° C. or less.

Temperature exposure may occur continuously, or at a frequency. For example, temperature exposure may occur at a frequency of about every hour, every 2 hours, every 4 hours, every 6 hours, every 8 hours, every 12 hours, every 24 hours, every 2 days, every 3 days, every week, or about every other week. Temperature exposure may occur at a frequency of at least about every hour, every 2 hours, every 4 hours, every 6 hours, every 8 hours, every 12 hours, every 24 hours, every 2 days, every 3 days, every week, or at least about every other week. Temperature exposure may occur at a frequency of no more than about every other week, every week, every 3 days, every 2 days, every 24 hours, every 12 hours, every 8 hours, every 6 hours, every 4 hours, every 3 hours, every 2 hours, or no more than about every hour. Temperature exposure treatments may be administered for a period of about 1 day, 2 days, 3 days, 4 days, 5 days, 6 days, 7 days, 10 days, 14 days, 21 days, 28 days, 1 month, 2 months, 3 months, 4 months, 5 months, or about 6 months. Temperature exposure treatments may be administered for a period of at least about 1 day, 2 days, 3 days, 4 days, 5 days, 6 days, 7 days, 10 days, 14 days, 21 days, 28 days, 1 month, 2 months, 3 months, 4 months, 5 months, or about 6 months. Temperature exposure may be administered for a period of no more than about 6 months, 5 months, 4 months, 3 months, 2 months, 1 month, 28 days, 21 days, 14 days, 10 days, 7 days, 6 days, 5 days, 4 days, 3 days, 2 days, or no more than about 1 day. Temperature exposure may be administered for a period in a range from about 1 day to about 7 days, about 1 day to about 14 days, about 1 day to about 21 days, about 1 day to about 28 days, about 1 day to about 1 month, about 1 day to about 3 months, about 1 day to about 6 months, about 7 days to about 14 days, about 7 days to about 21 days, about 7 days to about 28 days, about 7 days to about 1 month, about 7 days to about 3 months, about 7 days to about 6 months, about 14 days to about 21 days, about 14 days to about 28 days, about 14 days to about 1 month, about 14 days to about 3 months, about 14 days to about 6 months, about 21 days to about 28 days, about 21 days to about 1 month, about 21 days to about 3 months, about 21 days to about 6 months, about 28 days to about 1 month, about 28 days to about 3 months, about 28 days to about 6 months, about 1 month to about 3 months, about 1 month to about 6 months, or about 3 months to about 6 months.

Infrared radiation sources may be used as a method of creating heat exposure. An infrared radiation source may have a particular radiation wavelength. An infrared radiation source may have a wavelength of about 700 nm, 800 nm, 900 nm, 1 micrometer (μm), 5 μm, 10 μm, 50 μm, 100 μm, 250 μm, 500 μm, or about 1 mm. An infrared radiation source may have a wavelength of at least about 700 nm, 800 nm, 900 nm, 1 μm, 5 μm, 10 μm, 50 μm, 100 μm, 250 μm, 500 μm, or about 1 mm or more. An infrared radiation source may have a wavelength of no more than about 1 mm, 500 μm, 250 μm, 100 μm, 50 μm, 10 μm, 5 μm, 1 μm, 900 nm, 800 nm, or about 700 nm or less. An infrared radiation source may have a wavelength in a range from about 700 nm to about 1 μm, about 700 nm to about 5 μm, about 700 nm to about 10 μm, about 700 nm to about 50 μm, about 700 nm to about 100 μm, about 700 nm to about 250 μm, about 700 nm to about 500 μm, about 700 nm to about 1 mm, about 1 μm to about 5 μm, about 1 μm to about 10 μm, about 1 μm to about 50 μm, about 1 μm to about 100 μm, about 1 μm to about 250 μm, about 1 μm to about 500 μm, about 1 μm to about 1 mm, about 5 μm to about 10 μm, about 5 μm to about 50 μm, about 5 μm to about 100 μm, about 5 μm to about 250 μm, about 5 μm to about 500 μm, about 5 μm to about 1 mm, about 10 μm to about 50 μm, about 10 μm to about 100 μm, about 10 μm to about 250 μm, about 10 μm to about 500 μm, about 10 μm to about 1 mm, about 50 μm to about 100 μm, about 50 μm to about 250 μm, about 50 μm to about 500 μm, about 50 μm to about 1 mm, about 100 μm to about 250 μm, about 100 μm to about 500 μm, about 100 μm to about 1 mm, about 250 μm to about 500 μm, about 250 μm to about 1 mm, or from about 500 μm to about 1 mm.

Chemical exposure may be utilized as a method of creating one or more epigenetic modifications in cannabis plants. Chemical exposure may include the exposure of cannabis plants to any solid, liquid, or gaseous chemical that might create an abiotic stress in the cannabis plants. Chemicals may include toxins, mutagens, chemical or biological precursors, biological molecules, hormones, steroids, minerals, acids, bases, salts, and any others classes of chemical compounds that might produce a stress response in a cannabis plant. Chemical exposure may occur via infusion into the root system, application to foliage or other external parts of the plant, via gaseous infusion, or any other method. In some examples, a chemical exposure may comprise subjecting (e.g., contacting or administering) the cannabis plants with one or more DNA damaging chemicals. Examples of the DNA damaging chemicals can include, but are not limited to, benzene, hydroquinone, styrene, carbon tetrachloride, and trichloroethylene.

Chemical exposure may occur continuously, or at a frequency. For example, chemical exposure may occur at a frequency of about every hour, every 2 hours, every 4 hours, every 6 hours, every 8 hours, every 12 hours, every 24 hours, every 2 days, every 3 days, every week, or about every other week. Chemical exposure may occur at a frequency of at least about every hour, every 2 hours, every 4 hours, every 6 hours, every 8 hours, every 12 hours, every 24 hours, every 2 days, every 3 days, every week, or at least about every other week. Chemical exposure may occur at a frequency of no more than about every other week, every week, every 3 days, every 2 days, every 24 hours, every 12 hours, every 8 hours, every 6 hours, every 4 hours, every 3 hours, every 2 hours, or no more than about every hour. Chemical exposure treatments may be administered for a period of about 1 day, 2 days, 3 days, 4 days, 5 days, 6 days, 7 days, 10 days, 14 days, 21 days, 28 days, 1 month, 2 months, 3 months, 4 months, 5 months, or about 6 months. Chemical exposure treatments may be administered for a period of at least about 1 day, 2 days, 3 days, 4 days, 5 days, 6 days, 7 days, 10 days, 14 days, 21 days, 28 days, 1 month, 2 months, 3 months, 4 months, 5 months, or about 6 months. Chemical exposure may be administered for a period of no more than about 6 months, 5 months, 4 months, 3 months, 2 months, 1 month, 28 days, 21 days, 14 days, 10 days, 7 days, 6 days, 5 days, 4 days, 3 days, 2 days, or no more than about 1 day. Chemical exposure may be administered for a period in a range from about 1 day to about 7 days, about 1 day to about 14 days, about 1 day to about 21 days, about 1 day to about 28 days, about 1 day to about 1 month, about 1 day to about 3 months, about 1 day to about 6 months, about 7 days to about 14 days, about 7 days to about 21 days, about 7 days to about 28 days, about 7 days to about 1 month, about 7 days to about 3 months, about 7 days to about 6 months, about 14 days to about 21 days, about 14 days to about 28 days, about 14 days to about 1 month, about 14 days to about 3 months, about 14 days to about 6 months, about 21 days to about 28 days, about 21 days to about 1 month, about 21 days to about 3 months, about 21 days to about 6 months, about 28 days to about 1 month, about 28 days to about 3 months, about 28 days to about 6 months, about 1 month to about 3 months, about 1 month to about 6 months, or about 3 months to about 6 months.

The cultivation of cannabis plants, whether indoors or outdoors, may occur in a wide variety of soil compositions. Soil types for growth may include such types as alfisols, andisols, aridisols, entisols, gelisols, histosols, inceptisols, mollisols, oxisols, spodosols, ultisols, vertisols, or a combination of different types. Soil may comprise up to about 50% mineral matter, up to about 10% organic matter, up to about 30% water, and up to about 30% air. Soil may comprise minerals such as quartz (SiO₂), calcite (CaCO₃), feldspar (KAlSi₃O₈), dolomite (CaMg(CO₃)₂), gypsum (CaSO₄*2H₂O), alumina (AlOH₃), hematite (Fe₂O₃), iron hydroxide (FeOH₃), manganese bimessite (MnO₂), mica (KMg₃AlSiO₁₀OH₂), or biotite (KFe₃AlSi₃O₁₀OH₂). Soils may comprise mineral matter in the forms of gravel, sand, silt, or clay. Soils may comprise phosphorus compounds, nitrogen compounds, sulfur compounds, magnesium compounds, potassium compounds, calcium compounds, and sodium compounds. Soils may comprise clays such as alumino-silica clays (montmorillonite, illite, vermiculite, chlorite, kaolinite, or others), crystalline chain clays, amorphous clays, or sequioxide clays. Soils may comprise organic matter such as decomposed plant matter and microorganisms, such as bacteria and fungi.

A soil may have an optimum pH for plant growth, depending upon the type of soil and the plant species. An optimum soil pH may be about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or about 9.5. An optimal soil pH for plant growth may be no greater than about 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, or about 3.5. An optimal soil pH for plant growth may be no less than about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or about 9.5. An optimal soil pH for plant growth may occur in the range from about 3.5 to about 4.0, about 3.5 to about 4.5, about 3.5 to about 5.0, about 3.5 to about 5.5, about 3.5 to about 6.0, about 3.5 to about 6.5, about 3.5 to about 7.0, about 3.5 to about 7.5, about 3.5 to about 8.0, about 3.5 to about 8.5, about 3.5 to about 9.0, about 3.5 to about 9.5, about 4.0 to about 4.5, about 4.0 to about 5.0, about 4.0 to about 5.5, about 4.0 to about 6.0, about 4.0 to about 6.5, about 4.0 to about 7.0, about 4.0 to about 7.5, about 4.0 to about 8.0, about 4.0 to about 8.5, about 4.0 to about 9.0, about 4.0 to about 9.5, about 4.5 to about 5.0, about 4.5 to about 5.5, about 4.5 to about 6.0, about 4.5 to about 6.5, about 4.5 to about 7.0, about 4.5 to about 7.5, about 4.5 to about 8.0, about 4.5 to about 8.5, about 4.5 to about 9.0, about 4.5 to about 9.5, about 5.0 to about 5.5, about 5.0 to about 6.0, about 5.0 to about 6.5, about 5.0 to about 7.0, about 5.0 to about 7.5, about 5.0 to about 8.0, about 5.0 to about 8.5, about 5.0 to about 9.0, about 5.0 to about 9.5, about 5.5 to about 6.0, about 5.5 to about 6.5, about 5.5 to about 7.0, about 5.5 to about 7.5, about 5.5 to about 8.0, about 5.5 to about 8.5, about 5.5 to about 9.0, about 5.5 to about 9.5, about 6.0 to about 6.5, about 6.0 to about 7.0, about 6.0 to about 7.5, about 6.0 to about 8.0, about 6.0 to about 8.5, about 6.0 to about 9.0, about 6.0 to about 9.5, about 6.5 to about 7.0, about 6.5 to about 7.5, about 6.5 to about 8.0, about 6.5 to about 8.5, about 6.5 to about 9.0, about 6.5 to about 9.5, about 7.0 to about 7.5, about 7.0 to about 8.0, about 7.0 to about 8.5, about 7.0 to about 9.0, about 7.0 to about 9.5, about 7.5 to about 8.0, about 7.5 to about 8.5, about 7.5 to about 9.0, about 7.5 to about 9.5, about 8.0 to about 8.5, about 8.0 to about 9.0, about 8.0 to about 9.5, about 8.5 to about 9.0, about 8.5 to about 9.5, or about 9.0 to about 9.5.

The growth of cannabis plants may be supplemented by the addition of various factors that affect some aspect of plant development. Cannabis plants may be supplemented to increase their growth rate, increase their size, increase their hardiness or disease resistance, induce flowering, increase the yield of cannabinoids, or alter the composition profile of cannabinoids. In some aspects, a cannabis plant may be supplemented with olivetol, olivetolic acid, cannabigerol (CBG), geranyl pyrophosphate (GPP), or a combination of these compounds or others. The olivetol, olivetolic acid, CBG, or GPP supplementation may provide precursors to the cannabis plant to increase the expression of cannabinoids. The supplements may be provided in any manner that permits uptake of the supplement into the plant vasculature. In some aspects, a solution comprising olivetol, olivetolic acid, CBG, or GPP may be sprayed or misted on to the foliage, including leaves and flowering bodies. In other aspects, a solution comprising olivetol, olivetolic acid, CBG, or GPP may be watered in to the root system. Olivetol, olivetolic acid, CBG, or GPP may be provided to the cannabis plant at a sufficient concentration to enhance cannabinoid production. A supplement solution may comprise olivetol, olivetolic acid, or GPP at a concentration of no less than about 0.1 micromole per milliliter (μmol/ml), 0.5 μmol/ml, 1 μmol/ml, 5 μmol/ml, 10 μmol/ml, 50 μmol/ml, 100 μmol/ml, 250 μmol/ml, 500 μmol/ml, 1000 μmol/ml, 5000 μmol/ml, or no less than about 10000 μmol/ml. A supplement solution may comprise olivetol, olivetolic acid, CBG, or GPP at a concentration of no greater than about 10000 μmol/ml, 5000 μmol/ml, 1000 μmol/ml, 500 μmol/ml, 250 μmol/ml, 100 μmol/ml, 50 μmol/ml, 10 μmol/ml, 5 μmol/ml, 1 μmol/ml, 0.5 μmol/ml, or no greater than about 0.1 μmol/ml. A supplement solution comprising olivetol, olivetolic acid, CBG, or GPP may have a supplement concentration in the range from about 0.1 μmol/ml to about 0.5 μmol/ml, about 0.1 μmol/ml to about 1 μmol/ml, about 0.1 μmol/ml to about 5 μmol/ml, about 0.1 μmol/ml to about 10 μmol/ml, about 0.1 μmol/ml to about 50 μmol/ml, about 0.1 μmol/ml to about 100 μmol/ml, about 0.1 μmol/ml to about 250 μmol/ml, about 0.1 μmol/ml to about 500 μmol/ml, about 0.1 μmol/ml to about 1000 μmol/ml, about 0.1 μmol/ml to about 5000 μmol/ml, about 0.1 μmol/ml to about 10000 μmol/ml, about 0.5 μmol/ml to about 1 μmol/ml, about 0.5 μmol/ml to about 5 μmol/ml, about 0.5 μmol/ml to about 10 μmol/ml, about 0.5 μmol/ml to about 50 μmol/ml, about 0.5 μmol/ml to about 100 μmol/ml, about 0.5 μmol/ml to about 250 μmol/ml, about 0.5 μmol/ml to about 500 μmol/ml, about 0.5 μmol/ml to about 1000 μmol/ml, about 0.5 μmol/ml to about 5000 μmol/ml, about 0.5 μmol/ml to about 10000 μmol/ml, about 1 μmol/ml to about 5 μmol/ml, about 1 μmol/ml to about 10 μmol/ml, about 1 μmol/ml to about 50 μmol/ml, about 1 μmol/ml to about 100 μmol/ml, about 1 μmol/ml to about 250 μmol/ml, about 1 μmol/ml to about 500 μmol/ml, about 1 μmol/ml to about 1000 μmol/ml, about 1 μmol/ml to about 5000 μmol/ml, about 1 μmol/ml to about 10000 μmol/ml, about 5 μmol/ml to about 10 μmol/ml, about 5 μmol/ml to about 50 μmol/ml, about 5 μmol/ml to about 100 μmol/ml, about 5 μmol/ml to about 250 μmol/ml, about 5 μmol/ml to about 500 μmol/ml, about 5 μmol/ml to about 1000 μmol/ml, about 5 μmol/ml to about 5000 μmol/ml, about 5 μmol/ml to about 10000 μmol/ml, about 10 μmol/ml to about 50 μmol/ml, about 10 μmol/ml to about 100 μmol/ml, about 10 μmol/ml to about 250 μmol/ml, about 10 μmol/ml to about 500 μmol/ml, about 10 μmol/ml to about 1000 μmol/ml, about 10 μmol/ml to about 5000 μmol/ml, about 10 μmol/ml to about 10000 μmol/ml, about 50 μmol/ml to about 100 μmol/ml, about 50 μmol/ml to about 250 μmol/ml, about 50 μmol/ml to about 500 μmol/ml, about 50 μmol/ml to about 1000 μmol/ml, about 50 μmol/ml to about 5000 μmol/ml, about 50 μmol/ml to about 10000 μmol/ml, about 100 μmol/ml to about 250 μmol/ml, about 100 μmol/ml to about 500 μmol/ml, about 100 μmol/ml to about 1000 μmol/ml, about 100 μmol/ml to about 5000 μmol/ml, about 100 μmol/ml to about 10000 μmol/ml, about 250 μmol/ml to about 500 μmol/ml, about 250 μmol/ml to about 1000 μmol/ml, about 250 μmol/ml to about 5000 μmol/ml, about 250 μmol/ml to about 10000 μmol/ml, about 500 μmol/ml to about 1000 μmol/ml, about 500 μmol/ml to about 5000 μmol/ml, about 500 μmol/ml to about 10000 μmol/ml, about 1000 μmol/ml to about 5000 μmol/ml, about 1000 μmol/ml to about 10000 μmol/ml, or about 5000 μmol/ml to about 10000 μmol/ml.

A supplement such as olivetol, olivetolic acid, CBG, or GPP may be administered to a cannabis plant at a determined frequency for a determined amount of time. The supplement may be administered over the entire plant life. The supplement may be administered during specific phases of plant growth. The supplement may be given at a frequency of about every hour, every 2 hours, every 4 hours, every 6 hours, every 8 hours, every 12 hours, every 24 hours, every 2 days, every 3 days, every week, or about every other week. The supplement may be given at a frequency of at least about every hour, every 2 hours, every 4 hours, every 6 hours, every 8 hours, every 12 hours, every 24 hours, every 2 days, every 3 days, every week, or at least about every other week. The supplement may be given at a frequency of no more than about every other week, every week, every 3 days, every 2 days, every 24 hours, every 12 hours, every 8 hours, every 6 hours, every 4 hours, every 3 hours, every 2 hours, or no more than about every hour. The supplement may be administered for a period of about 1 day, 2 days, 3 days, 4 days, 5 days, 6 days, 7 days, 10 days, 14 days, 21 days, 28 days, 1 month, 2 months, 3 months, 4 months, 5 months, or about 6 months. The supplement may be administered for a period of at least about 1 day, 2 days, 3 days, 4 days, 5 days, 6 days, 7 days, 10 days, 14 days, 21 days, 28 days, 1 month, 2 months, 3 months, 4 months, 5 months, or about 6 months. The supplement may be administered for a period of no more than about 6 months, 5 months, 4 months, 3 months, 2 months, 1 month, 28 days, 21 days, 14 days, 10 days, 7 days, 6 days, 5 days, 4 days, 3 days, 2 days, or no more than about 1 day. A supplement of olivetol, olivetolic acid, or GPP may be administered for a period in a range from about 1 day to about 7 days, about 1 day to about 14 days, about 1 day to about 21 days, about 1 day to about 28 days, about 1 day to about 1 month, about 1 day to about 3 months, about 1 day to about 6 months, about 7 days to about 14 days, about 7 days to about 21 days, about 7 days to about 28 days, about 7 days to about 1 month, about 7 days to about 3 months, about 7 days to about 6 months, about 14 days to about 21 days, about 14 days to about 28 days, about 14 days to about 1 month, about 14 days to about 3 months, about 14 days to about 6 months, about 21 days to about 28 days, about 21 days to about 1 month, about 21 days to about 3 months, about 21 days to about 6 months, about 28 days to about 1 month, about 28 days to about 3 months, about 28 days to about 6 months, about 1 month to about 3 months, about 1 month to about 6 months, or about 3 months to about 6 months.

Cannabinoids disclosed herein include but are not limited to cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C₄ (CBN-C₄), cannabinol-C₂ (CBN-C₂), cannabiorcol (CBN-C₁), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), and cannabiripsol (CBR) cannabinoids.

The cannabinoids of the subject compositions disclosed herein can comprise cannabidiol-class compounds, including but not limited to cannabidiol (CBD), cannabidiolic acid (CBDA), cannabidiol monomethylether (CBDM), cannabidiol-C₄ (CBD-C₄), cannabidivarin (CBDV), cannabidivarinic acid (CBDVA), cannabidiorcol (CBD-C₁), and combinations thereof. CBD can comprise delta-1-cannabidiol, delta-2-cannabidiol, delta-3-cannabidiol, delta-3,7-cannabidiol, delta-4-cannabidiol, delta-5-cannabidiol, delta-6-cannabidiol, and combinations thereof.

The compositions of the present disclosure can comprise tetrahydrocannabinol (THC) as a type of cannabinoids. THC can comprise delta-9-THC, delta-8-THC, and combinations thereof. THC can comprise delta-6a,7-tetrahydrocannabinol, delta-7-tetrahydrocannabinol, delta-8-tetrahydrocannabinol, delta-9,11-tetrahydrocannabinol, delta-9-tetrahydrocannabinol, delta-10-tetrahydrocannabinol, delta-6a,10a-tetrahydrocannabinol, and combinations thereof. Delta-9-tetrahydrocannabinol can comprise stereoisomers including (6aR,10aR)-delta-9-tetrahydrocannabinol, (6aS,10aR)-delta-9-tetrahydrocannabinol, (6aS,10aS)-delta-9-tetrahydrocannabinol, (6aR,10aS)-delta-9-tetrahydrocannabinol, and combinations thereof.

The compositions of the present disclosure can comprise one or more terpene compounds, including but not limited to terpenoids such as monoterpenoids, sesquiterpenoids, diterpenoids, and triterpenoids. Terpenes can be acyclic, monocyclic, or polycyclic. Terpenes can include but are not limited to myrcene, limonene, linalool, trans-ocimene, cis-ocimene, alpha-pinene, beta-pinene, alpha-humulene (alpha-caryophyllene), beta-caryophyllene, delta-3-carene, trans-gamma-bisabolene, cis-gamma-bisabolene, trans-alpha-farnesene, cis-beta-farnesene, beta-fenchol, beta-phellandrene, guajol, alpha-gualene, alpha-eudesmol, beta-eudesmol, gamma-eudesmol, terpinolene, alpha-selinene, beta-selinene, alpha-terpineol, fenchone, camphene, cis-sabinene hydrate, alpha-trans-bergamotene, alpha-cis-bergamotene, borneol, gamma-curcumene, alpha-thujene, epi-alpha-bisabolol, ipsdienol, alpha-ylangene, beta-elemene, gamma-muurolene, alpha-cadinene, alpha-longipinene, caryophyllene oxide, and combinations thereof.

Cannabis plants may be harvested and processed in a modular fashion. A modular cannabis production process may involve one or more units that carry out various tasks to prepare cannabis plants for processing and then perform subsequent processing steps to produce useful products. A modular cannabis production process may be a zero-waste process where all cannabis plant components are utilized by some process. A modular cannabis production process may include, but is not limited to, units for harvesting, separating biomass components (e.g. separating the flowering bodies from the stalk), decorticating the stalk, shredding, grinding, chipping, or milling certain components, performing cannabinoid extractions, converting cannabis biomass into biofuels or bioenergy, and processing biomass into exportable materials. The modular cannabis processing system may comprise other units, such as an inducing unit (e.g., UV exposure unit, acoustic exposure unit, solution exposure unit, etc.), as described elsewhere herein. The cannabis processing system may comprise at least one, two, three, four, five, six, seven, eight, nine, or ten or more of the modular processing units (and/or functions) described above. In some instances, two or more processing units may be integral or combined as a sub-assembly unit within the modular cannabis processing system. In some instances, a processing unit may comprise a plurality of sub-units (e.g., in parallel or in series) that can, individually or in combination, achieve the function of the processing unit. In some instances, two or more processing units (e.g., a decortication unit and one or more additional processing units disclosed herein) may be combined in a continuous processing unit within the modular cannabis processing system. The continuous processing unit may be configured to operate automatically without human intervention. Alternatively, the continuous processing unit may be operable with human intervention. The two or more processing units of the modular cannabis processing system may be releasably coupled to each other. The order of the two or more processing units in the processing procedure of the modular cannabis processing system may be changed. Alternatively, the order of the two or more processing units in the processing procedure of the modular cannabis processing system may be permanent. The two or more processing units may be coupled to each other via one or more transport units. Examples of the one or more transport units may include, but are not limited to, a roller (e.g., conveying roller), a belt (e.g., a conveyor belt, a treadmill belt, etc.), a chain (e.g., a conveying chain), a chute, and/or a pulley. The one or more transport units may be operatively coupled to one or more actuators to direct a transport of an object (e.g., at least a portion of a plant, such as a Cannabis plant, as disclosed herein) from one processing unit to another processing unit. Examples of the one or more actuators may include, but are not limited to, a mechanical, hydraulic, pneumatic, or electro-mechanical actuator. In some examples, the one or more transport units may be a robot, such as an automated robot.

One or more processing units as provided herein may be operatively coupled (e.g., communicatively coupled to) to a controller. The controller may be configured to direct operation of the one or more processing units during the processing (e.g., the continuous processing) of the plant (e.g., the Cannabis plant). The operation may be decortication, foliage removal, de-gumming, trichome collection, seed collection, seed processing, bast fiber collection, bast fiber processing, bundling residual biomass, cannabinoid extraction, biomass gasification, biochar production, and/or biomass palletization. The controller may be operatively coupled (e.g., communicatively coupled to) the one or more transport units. The controller may be configured to direct the one or more transport units to transport at least a portion (e.g., a processed portion) of the plant (e.g., the Cannabis plant) from one processing unit to another processing unit.

A modular cannabis production system may output such usable commodities as electrical energy, biochar, ash fertilizer, syngas, oils, waxes, seeds, seed hulls, liquid hydrocarbons, bast fibers, building materials (e.g. particle board), fuel pellets, bundled stalks, trichomes, and cannabis-derived edibles such as oils, extracts, and food products. A modular cannabis production process may be mobile for field deployment or stationary. A modular cannabis production process may contain some or all of the above-described processes.

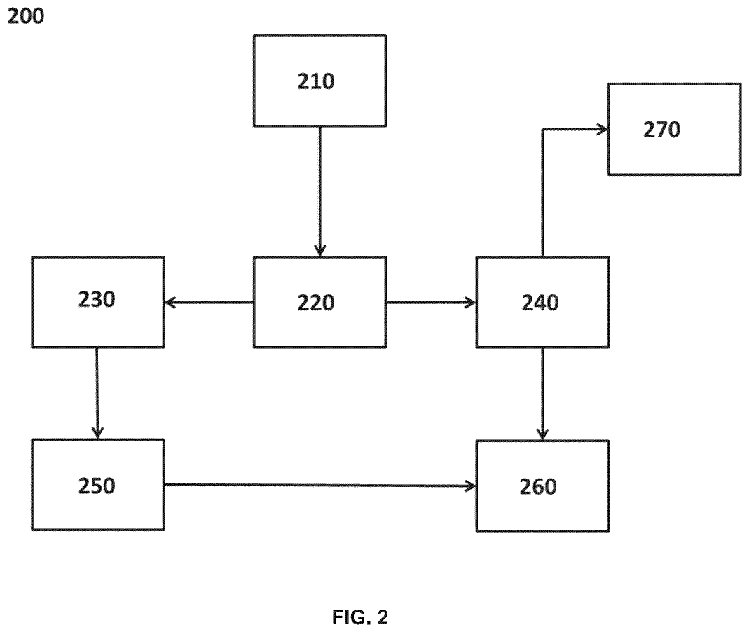

FIG. 2 shows a possible modular cannabis production process 200. Harvested cannabis plants from a cannabis growth operation 210 may be sent to a rendering operation 220 to be divided into individual components such as flowering bodies, foliage, seeds, and stalks. Flowering bodies may be sent to a trichome recovery process 230, and trichomes may be subsequently sent to an extraction process 250 to synthesize CBD oils or other cannabinoid compositions. Any residual biomass from an extraction process may be sent to a biofuel production process 260, such as biomass gasification or biochar production. Stalks and other cannabis components may be sent to a decortication and degumming process 230 to collect bast fibers. Residual biomass components after decortication may be sent to a biofuel production process 260. Residual biomass components after decortication may also be sent to other processes 270 such as bundling and bailing or building material production processes (e.g. particle board or insulation).

A modular cannabis production process may comprise a unit that captures the flowering body or any components of the flowering body. The unit may cut or render the flowering body from the stalk or the flowering bodies may be manually removed and fed into the unit. The flowering body unit may further process the flowering body to separate the most valuable constituents from the flowering body. The processing unit may actively separate trichomes via cutting or another approach of mechanical separation to isolate and collect the trichomes. Trichomes may comprise small, glandular bodies of approximately 20 μm to 30 μm in diameter and length. Mesh filters, netting, sifters, or other devices may be used to capture trichomes. Trichomes may be passively separated and collected during the processing of the flowering body. In some aspects, a trichome capture unit may be configured to capture passively lost trichomes. In other aspects, a trichome capture unit may be configured to actively sift trichomes away from other materials to create a highly selective trichome isolation process.

Sifters, filters, or netting may be sized to adequately capture or pass trichomes. For example, a sifter, filter or net may have an average mesh particle size of no less than about 1 μm, 2 μm, 3 μm, 4 μm, 5 μm, 6 μm, 7 μm, 8 μm, 9 μm, 10 μm, 11 μm, 22 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 60 μm, 70 μm, 80 μm, 90 μm, or no less than about 100 μm. A sifter, filter or net may have an average mesh particle size of no greater than about 100 μm, 90 μm, 80 μm, 70 μm, 60 μm, 50 μm, 49 μm, 48 μm, 47 μm, 46 μm, 45 μm, 44 μm, 43 μm, 42 μm, 41 μm, 40 μm, 39 μm, 38 μm, 37 μm, 36 μm, 35 μm, 34 μm, 33 μm, 32 μm, 31 μm, 30 μm, 29 μm, 28 μm, 27 μm, 26 μm, 25 μm, 24 μm, 23 μm, 22 μm, 21 μm, 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm, 4 μm, 3 μm, 2 μm, or no greater than about 1 μm. A sifter, filter, or net may have a mesh particle size in a range from about 1 μm to about 5 μm, about 1 μm to about 10 μm, about 1 μm to about 15 μm, about 1 μm to about 20 μm, about 1 μm to about 25 μm, about 1 μm to about 30 μm, about 1 μm to about 35 μm, about 1 μm to about 40 μm, about 1 μm to about 45 μm, about 1 μm to about 50 μm, about 1 μm to about 100 μm, about 5 μm to about 10 μm, about 5 μm to about 15 μm, about 5 μm to about 20 μm, about 5 μm to about 25 μm, about 5 μm to about 30 μm, about 5 μm to about 35 μm, about 5 μm to about 40 μm, about 5 μm to about 45 μm, about 5 μm to about 50 μm, about 5 μm to about 100 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 10 μm to about 100 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 15 μm to about 100 μm, about 20 μm to about 25 μm, about 20 μm to about 30 μm, about 20 μm to about 35 μm, about 20 μm to about 40 μm, about 20 μm to about 45 μm, about 20 μm to about 50 μm, about 20 μm to about 100 μm, about 25 μm to about 30 μm, about 25 μm to about 35 μm, about 25 μm to about 40 μm, about 25 μm to about 45 μm, about 25 μm to about 50 μm, about 25 μm to about 100 μm, about 30 μm to about 35 μm, about 30 μm to about 40 μm, about 30 μm to about 45 μm, about 30 μm to about 50 μm, about 30 μm to about 100 μm, about 35 μm to about 40 μm, about 35 μm to about 45 μm, about 35 μm to about 50 μm, about 35 μm to about 100 μm, about 40 μm to about 45 μm, about 40 μm to about 50 μm, about 40 μm to about 100 μm, about 45 μm to about 50 μm, about 45 μm to about 100 μm, or about 50 μm to about 100 μm.

In some cases, flowering bodies may comprise seeds. A modular cannabis production process may comprise one or more units to separate cannabis seeds from a flowering body and further process the seeds. Further processes may include cleaning the seeds of residual material, shelling the seeds, press extracting the seeds to create hemp seed oil, processing the seed hulls into surfactants or other useful materials, packaging the seeds for export, heating the seeds to reduce their viability, and packaging the seeds for consumption.

A modular cannabis production process may include a unit for decortication. Decortication also provides any process that separates plant fibers from the remainder of the plant structure. Cannabis plants may comprise bast fibers, especially in the stalk. Bast fibers may be decorticated from the cannabis stalk by any process that allows effective isolation of the fibers from the stalk. A decortication process may comprise a mechanical separation process. Mechanical decortication may be performed in a mobile decortication unit or in a stationary processing facility. Mechanical decortication may involve disrupting the stalk structure via blades, rollers, or other mechanisms of cutting or crushing. Mechanical decortication may occur using dried or freshly-harvested cannabis plants. Decortication may also comprise a wet method. Wet decortication may comprise enzymatic or microorganism-influenced processes that cause the breakdown of the structure of the cannabis biomass, leading to the release of the bast fibers. Decortication may require a degumming process for completion. Degumming may involve the removal of residual biomass components such as pectins and lignins to allow full release of the fiber strands.

A modular cannabis production process may require a step comprising a degumming process. The degumming process may occur as a part of the decortication process when producing bast fibers. The degumming process may remove pectins or other biomass components that bind recoverable bast fibers. Many methods may be employed for plant fiber degumming that allow for separation of fiber from the woody part, and removal of non-cellulosic components such as pectin, hemicellulose, lignin, waxes and fats, such as dew retting of fibrous plants in the field. Dew retting may occur immediately after harvest in the area where the cannabis plants were grown or in another area. Dew retting may utilize microorganisms, including fungi, which penetrate the swathed stems and decompose pectin, a plant glue of the fibrous content, with enzymes and thus conduct the process. Such a process may yield a fiber of a quality that depends upon the atmospheric conditions (air temperature, humidity, rain) in which the fiber is obtained. Dew retting may occur passively or may be assisted by the active application of degumming agents. Degumming agents may include solutions comprising bacterial or fungal species. Applied microorganisms may be natural strains or genetically-modified strains for enhanced digestion of biomass components such as pectins, lignins, waxes, and fats. A dew retting process may be allowed to occur for a period of at least about 1 day, 2 days, 3 days, 4, days, 5 days, 6 days, 7 days, 8 days, 9 days, 10 days, 11 day, 12 days, 13 days, 14, days, 15 days, 16 days, 17 days, 18 days, 19 days, 20 days, 21 day, 22 days, 23 days, 24, days, 25 days, 26 days, 27 days, 28 days, 29 days, or at least about 30 days. A dew retting process may be limited to occurring for a period of no longer than about 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day.

Another method of fibrous plant degumming may include water retting in special tanks or ponds. In some aspects, water retting may involve any biochemical phenomena occurring as result of bacteria fermentation that causes the separation of the woody part of the stem from the fiber. In other aspects, water retting may involve the controlled application of enzymes that specifically digest particular biomass components such as pectins, lignins, waxes, and fats. Microorganisms or enzymes utilized during water retting may comprise naturally-occurring strains or genetically-modified strains. Water retting may be conducted in aerobic and anaerobic conditions. Water retting may occur under controlled, regulated, or optimized conditions. Water retting process variables may include temperature, pH, and oxygen partial pressure. The temperature of a water retting process may be no less than about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or no less than about 50° C. The temperature of a water retting process may be no greater than about 50° C., 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., or no greater than about 10° C. A water retting process may occur within a temperature range of about 10° C. to about 15° C., about 10° C. to about 20° C., about 10° C. to about 25° C., about 10° C. to about 30° C., about 10° C. to about 35° C., about 10° C. to about 40° C., about 10° C. to about 45° C., about 10° C. to about 50° C., about 15° C. to about 20° C., about 15° C. to about 25° C., about 15° C. to about 30° C., about 15° C. to about 35° C., about 15° C. to about 40° C., about 15° C. to about 45° C., about 15° C. to about 50° C., about 20° C. to about 25° C., about 20° C. to about 30° C., about 20° C. to about 35° C., about 20° C. to about 40° C., about 20° C. to about 45° C., about 20° C. to about 50° C., about 25° C. to about 30° C., about 25° C. to about 35° C., about 25° C. to about 40° C., about 25° C. to about 45° C., about 25° C. to about 50° C., about 30° C. to about 35° C., about 30° C. to about 40° C., about 30° C. to about 45° C., about 30° C. to about 50° C., about 35° C. to about 40° C., about 35° C. to about 45° C., about 35° C. to about 50° C., about 40° C. to about 45° C., about 40° C. to about 50° C., or about 45° C. to about 50° C.

The reactivity of a water retting process may be controlled using acids, bases, or buffers. A water retting process may have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. A water retting process may have a pH of no greater than about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, or about 4.0. A water retting process may have a pH of no less than about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0. A water retting process may occur in a pH range from about 4.0 to about 4.5, about 4.0 to about 5.0, about 4.0 to about 5.5, about 4.0 to about 6.0, about 4.0 to about 6.5, about 4.0 to about 7.0, about 4.0 to about 7.5, about 4.0 to about 8.0, about 4.0 to about 8.5, about 4.0 to about 9.0, about 4.5 to about 5.0, about 4.5 to about 5.5, about 4.5 to about 6.0, about 4.5 to about 6.5, about 4.5 to about 7.0, about 4.5 to about 7.5, about 4.5 to about 8.0, about 4.5 to about 8.5, about 4.5 to about 9.0, about 5.0 to about 5.5, about 5.0 to about 6.0, about 5.0 to about 6.5, about 5.0 to about 7.0, about 5.0 to about 7.5, about 5.0 to about 8.0, about 5.0 to about 8.5, about 5.0 to about 9.0, about 5.5 to about 6.0, about 5.5 to about 6.5, about 5.5 to about 7.0, about 5.5 to about 7.5, about 5.5 to about 8.0, about 5.5 to about 8.5, about 5.5 to about 9.0, about 6.0 to about 6.5, about 6.0 to about 7.0, about 6.0 to about 7.5, about 6.0 to about 8.0, about 6.0 to about 8.5, about 6.0 to about 9.0, about 6.5 to about 7.0, about 6.5 to about 7.5, about 6.5 to about 8.0, about 6.5 to about 8.5, about 6.5 to about 9.0, about 7.0 to about 7.5, about 7.0 to about 8.0, about 7.0 to about 8.5, about 7.0 to about 9.0, about 7.5 to about 8.0, about 7.5 to about 8.5, about 7.5 to about 9.0, about 8.0 to about 8.5, about 8.0 to about 9.0, or about 8.5 to about 9.0.

The oxygen concentration of the water in a water retting process may be controlled to create aerobic or anaerobic fermentation conditions. The dissolved oxygen concentration may be less than about 10 milligrams per liter (mg/L), 9 mg/L, 8 mg/L, 7 mg/L, 6 mg/L, 5 mg/L, 4 mg/L, 3, mg/L, 2 mg/L, 1 mg/L, 0.9 mg/L, 0.8 mg/L, 0.7 mg/L, 0.6 mg/L, 0.5 mg/L, 0.4 mg/L, 0.3, mg/L, 0.2 mg/L, 0.1 mg/L, 0.09 mg/L, 0.08 mg/L, 0.07 mg/L, 0.06 mg/L, 0.05 mg/L, 0.04 mg/L, 0.03, mg/L, 0.02 mg/L, 0.01 mg/L, 0.009 mg/L, 0.008 mg/L, 0.007 mg/L, 0.006 mg/L, 0.005 mg/L, 0.004 mg/L, 0.003, mg/L, 0.002 mg/L, or less than about 0.001 mg/L. The dissolved oxygen concentration may be greater than about 0.001 mg/L, 0.002 mg/L, 0.003 mg/L, 0.004 mg/L, 0.005 mg/L, 0.006 mg/L, 0.007 mg/L, 0.008 mg/L, 0.009 mg/L, 0.01 mg/L, 0.02 mg/L, 0.03 mg/L, 0.04 mg/L, 0.05 mg/L, 0.06 mg/L, 0.07 mg/L, 0.08 mg/L, 0.09 mg/L, 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 2 mg/L, 3 mg/L, 4 mg/L, 5 mg/L, 6 mg/L, 7 mg/L, 8 mg/L, 9 mg/L, or greater than about 10 mg/L.

Cannabis-derived fibers, such as bast fibers, may be further processed or upgraded through a variety of processes. Bast fibers may be processed into textiles, ropes, twines, yarns, or threads. A modular cannabis production process may include one or more units for the upgrading of cannabis-derived fibers. Such units may perform upgrading processes such as delignification, cleaning, blending, mixing, scutching, carding, combing, drawing, spinning, checking, folding, twisting, plying, gassing, weaving, winding, warping, sizing, looming, pirning, knitting, desizing, scouring, bleaching, mercerising, singeing, raising, calendering, shrinking, and dyeing. A modular cannabis production process may produce a finished textile or other product, or any intermediate for the production of textiles or other products.

A modular cannabis production process may utilize residual cannabis-derived biomass components in various ways. In some aspects, residual cannabis biomass may be bailed and bundled. Bailed cannabis biomass may be exported to a different location for numerous purposes including biofuel generation, composting, extraction of cannabinoids, biochemical conversion, or the creation of materials such as insulation or particle-board. In other aspects, cannabis-derived biomass may be utilized in a modular production process that completely processes the cannabis plant in a single location. The stationary modular process may include biofuel generation, composting, extraction of cannabinoids, biochemical conversion, or the creation of materials such as insulation or particle-board. The modular processing equipment may be located in a single, fixed location with cannabis-derived biomass brought in and stored at the central location. The modular processing equipment may be portable or mobile such that it may be repositioned directly where the harvesting of plants is occurring.

A cannabis processing method may comprise a freezing process for harvested plant materials. Frozen plant materials may include any portion of a cannabis plant, including the flowering bodies and seeds. Freezing may be used as a method for preserving plant materials for later processing steps. Freezing may prevent the degradation, oxidation, or further reaction certain cannabinoids within the plant material. A freezing process may be used to alter the amounts of specific cannabinoids present within a portion of a cannabis plant. A freezing process may comprise a flash freezing method. A freezing process may occur in a refrigeration unit. A freezing process may utilize a medium such as dry ice (frozen carbon dioxide) to achieve rapid cooling. Frozen cannabis plant materials may be frozen as the first step of a cryogenic processing method (e.g. cryogenic extraction).

Freezing may extend the shelf-life of cannabis plant materials, allowing them to be preserved for later processing. A particular frozen cannabis plant material may have a shelf-life of about 1 week, 2 weeks, 1 month, 3 months, 6 months, 1 year, 2 years, 5 years or more. A particular frozen cannabis plant material may have a shelf-life of at least about 1 week, 2 weeks, 1 month, 3 months, 6 months, 1 year, 2 years, 5 years or more. A particular frozen cannabis plant material may have a shelf-life of no more than about 5 years, 2 years, 1 year, 6 months, 3 months, 1 month, 2 weeks, 1 week or less.

A freezing process for cannabis plant materials may occur at a particular temperature. Cannabis plant materials may be frozen at about 0° C., −10° C., −20° C., −30° C., −40° C., −50° C., −60° C., −70° C., or about −80° C. Cannabis plant materials may be frozen at a temperature of at least about −80° C., −70° C., −60° C., −50° C., −40° C., −30° C., −20° C., −10° C., or at least about 0° C. or more. Cannabis plant materials may be frozen at no more than a temperature of about 0° C., −10° C., −20° C., −30° C., −40° C., −50° C., −60° C., −70° C., or about −80° C. or less. Cannabis plant materials may be frozen in a temperature range from about 0° C. to about −10° C., about 0° C. to about −20° C., about 0° C. to about −40° C., about 0° C. to about −60° C., about 0° C. to about −80° C., about −10° C. to about −20° C., about −10° C. to about −40° C., about −10° C. to about −60° C., about −10° C. to about −80° C., about −20° C. to about −40° C., about −20° C. to about −60° C., about −20° C. to about −80° C., about −40° C. to about −60° C., about −40° C. to about −80° C., or about −60° C. to about −80° C.

To prepare cannabis-derived biomass for further processing, a modular cannabis production process may comprise additional rendering steps such as shredding, chipping, grinding, and milling. These processes may be conducted in batch processes or in continuous processes. Residual biomass may pass through one or more of these processes during the preparation process. Biomass may be rendered for further processing after drying or with full moisture content depending upon the requirements of the process. Biomass may be rendered to a particular characteristic size depending upon the application. In some aspects, larger but thin pieces or slices may be required to produce a building material such as particle board. In other aspects, finer shreds or powders may be required to pelletize the biomass when creating fuel pellets. Rendered biomass may have a critical characteristic size of no greater than about 10 centimeters (cm), 5 cm, 1 cm, 5 mm, 1 mm, 500 μm, 250 μm, 100 μm, 50 μm, 10 μm, or no greater than about 1 μm. Rendered biomass may have a critical characteristic size of no less than about 1 μm, 10 μm, 50 μm, 100 μm, 250 μm, 500 μm, 1 mm, 5 mm, 1 cm, 5 cm, or no less than about 10 cm. A biomass rendering process may produce biomass particles in a range from about 1 μm to about 10 μm, about 1 μm to about 50 μm, about 1 μm to about 100 μm, about 1 μm to about 250 μm, about 1 μm to about 500 μm, about 1 μm to about 1 mm, about 1 μm to about 5 mm, about 1 μm to about 1 cm, about 1 μm to about 5 cm, about 1 μm to about 10 cm, about 10 μm to about 50 μm, about 10 μm to about 100 μm, about 10 μm to about 250 μm, about 10 μm to about 500 μm, about 10 μm to about 1 mm, about 10 μm to about 5 mm, about 10 μm to about 1 cm, about 10 μm to about 5 cm, about 10 μm to about 10 cm, about 50 μm to about 100 μm, about 50 μm to about 250 μm, about 50 μm to about 500 μm, about 50 μm to about 1 mm, about 50 μm to about 5 mm, about 50 μm to about 1 cm, about 50 μm to about 5 cm, about 50 μm to about 10 cm, about 100 μm to about 250 μm, about 100 μm to about 500 μm, about 100 μm to about 1 mm, about 100 μm to about 5 mm, about 100 μm to about 1 cm, about 100 μm to about 5 cm, about 100 μm to about 10 cm, about 250 μm to about 500 μm, about 250 μm to about 1 mm, about 250 μm to about 5 mm, about 250 μm to about 1 cm, about 250 μm to about 5 cm, about 250 μm to about 10 cm, about 500 μm to about 1 mm, about 500 μm to about 5 mm, about 500 μm to about 1 cm, about 500 μm to about 5 cm, about 500 μm to about 10 cm, about 1 mm to about 5 mm, about 1 mm to about 1 cm, about 1 mm to about 5 cm, about 1 mm to about 10 cm, about 5 mm to about 1 cm, about 5 mm to about 5 cm, about 5 mm to about 10 cm, about 1 cm to about 5 cm, about 1 cm to about 10 cm, or about 5 cm to about 10 cm.

A modular cannabis production process may include one or more units that convert residual cannabis-derived biomass into biofuels or bioenergy. Biofuel conversion processes may include biomass gasification, biomass pelletization, biochar production, and biomass oil production. Any residual biomass from the cannabis harvesting and processing process may be available to a biofuel generation process. Potential sources of cannabis-derived biofuels may include roots, stems, leaves, flowering bodies, bark, and hurd, as well as any residual materials from other utilization processes, such as cannabinoid extraction. Different types of cannabis-derived biomass may be utilized in different processes depending upon the fuel characteristics. Biomass fuels may be characterized by their volatile matter content, ash content, heating value, and moisture content.

Cannabis-derived biomass may have a volatile matter content depending upon its chemical composition. Volatile matter content may be defined by the weight of matter released from a solid during non-oxidative heating. Volatile matter may be expressed as a percentage of the initial dry mass of an unheated solid. A cannabis-derived biomass component may have a volatile matter content of no less than about 10%, 15%, 20%, 25%, 30%, 35%, 40%, 45%, 50%, 55%, 60%, 65%, 70%, 75%, 80%, 85%, or about 90%. A cannabis-derived biomass component may have a volatile matter content of no greater than about 90%, 85%, 80%, 75%, 70%, 65%, 60%, 55%, 50%, 45%, 40%, 35%, 30%, 25%, 20%, 15%, or no greater than about 10%.

Cannabis-derived biomass may have an ash content depending upon its chemical composition. Ash content may be defined as the weight of matter remaining after a complete combustion reaction of a solid fuel. Ash may comprise inherent biomass constituents such as salts and minerals. Ash content may also derive from residual materials such as sand, dust, and dirt that become deposited on plants during agricultural processes. Ash content may be expressed as a percentage of the initial dry mass of an unheated solid. A cannabis-derived biomass component may have an ash content of no less than about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or no less than about 30%. A cannabis-derived biomass component may have an ash content of no less than about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%. Ash content may vary from one biomass component to another. Ash content may vary within a single component. Ash content for a particular cannabis-derived biomass component may occur in a range from about 0.1% to about 0.5%, about 0.1% to about 1%, about 0.1% to about 5%, about 0.1% to about 10%, about 0.1% to about 15%, about 0.1% to about 20%, about 0.1% to about 25%, about 0.1% to about 30%, about 0.5% to about 1%, about 0.5% to about 5%, about 0.5% to about 10%, about 0.5% to about 15%, about 0.5% to about 20%, about 0.5% to about 25%, about 0.5% to about 30%, about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%.

The moisture content of cannabis-derived biomass may vary depending upon the type of biomass and the conditions such as time since harvest. Cannabis-derived biomass may have an initial moisture content of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or about 70% on a weight basis. A modular cannabis production process may incorporate drying processes including ovens, kilns, or solar-heated storage. A drying process may reduce the biomass moisture content to a determined level. A drying process may reduce the moisture content of the cannabis-derived biomass by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or at least about 99%. Cannabis-derived biomass may have a final moisture content before a processing step of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% on a weight basis. Cannabis-derived biomass may have a final moisture content before a processing step of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% on a weight basis. Cannabis-derived biomass may have a final moisture content before a processing step of no more than about 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or no more than about 1% on a weight basis.

A cannabis-derived biomass component may have a particular heating value. The heating value may be defined as the energy released by complete combustion of the material on a per weight basis. The heating values of different components of a cannabis plant may vary. The heating value of a particular component of a cannabis plant may vary due to various factors such as species and growing conditions. A cannabis-derived biomass component may have a higher heating value of at least about 10 megaJoules per kilogram (MJ/kg), 20 MJ/kg, 30 MJ/kg, 40 MJ/kg, 50 MJ/kg, 60 MJ/kg, 70 MJ/kg, 80 MJ/kg, 90 MJ/kg, 100 MJ/kg, 120 MJ/kg, 140 MJ/kg, 160 MJ/kg, 180 MJ/kg, or at least about 200 MJ/kg. A cannabis-derived biomass component may have a higher heating value of no greater than about 200 MJ/kg, 180 MJ/kg, 160 MJ/kg, 140 MJ/kg, 120 MJ/kg, 100 MJ/kg, 90 MJ/kg, 80 MJ/kg, 70 MJ/kg, 60 MJ/kg, 50 MJ/kg, 40 MJ/kg, 30 MJ/kg, 20 MJ/kg, or no greater than about 10 MJ/kg. A cannabis-derived biomass component may have a higher heating value in a range from about 10 MJ/kg to about 20 MJ/kg, about 10 MJ/kg to about 40 MJ/kg, about 10 MJ/kg to about 60 MJ/kg, about 10 MJ/kg to about 80 MJ/kg, about 10 MJ/kg to about 100 MJ/kg, about 10 MJ/kg to about 120 MJ/kg, about 10 MJ/kg to about 140 MJ/kg, about 10 MJ/kg to about 160 MJ/kg, about 10 MJ/kg to about 180 MJ/kg, about 10 MJ/kg to about 200 MJ/kg, about 20 MJ/kg to about 40 MJ/kg, about 20 MJ/kg to about 60 MJ/kg, about 20 MJ/kg to about 80 MJ/kg, about 20 MJ/kg to about 100 MJ/kg, about 20 MJ/kg to about 120 MJ/kg, about 20 MJ/kg to about 140 MJ/kg, about 20 MJ/kg to about 160 MJ/kg, about 20 MJ/kg to about 180 MJ/kg, about 20 MJ/kg to about 200 MJ/kg, about 40 MJ/kg to about 60 MJ/kg, about 40 MJ/kg to about 80 MJ/kg, about 40 MJ/kg to about 100 MJ/kg, about 40 MJ/kg to about 120 MJ/kg, about 40 MJ/kg to about 140 MJ/kg, about 40 MJ/kg to about 160 MJ/kg, about 40 MJ/kg to about 180 MJ/kg, about 40 MJ/kg to about 200 MJ/kg, about 60 MJ/kg to about 80 MJ/kg, about 60 MJ/kg to about 100 MJ/kg, about 60 MJ/kg to about 120 MJ/kg, about 60 MJ/kg to about 140 MJ/kg, about 60 MJ/kg to about 160 MJ/kg, about 60 MJ/kg to about 180 MJ/kg, about 60 MJ/kg to about 200 MJ/kg, about 80 MJ/kg to about 100 MJ/kg, about 80 MJ/kg to about 120 MJ/kg, about 80 MJ/kg to about 140 MJ/kg, about 80 MJ/kg to about 160 MJ/kg, about 80 MJ/kg to about 180 MJ/kg, about 80 MJ/kg to about 200 MJ/kg, about 100 MJ/kg to about 120 MJ/kg, about 100 MJ/kg to about 140 MJ/kg, about 100 MJ/kg to about 160 MJ/kg, about 100 MJ/kg to about 180 MJ/kg, about 100 MJ/kg to about 200 MJ/kg, about 120 MJ/kg to about 140 MJ/kg, about 120 MJ/kg to about 160 MJ/kg, about 120 MJ/kg to about 180 MJ/kg, about 120 MJ/kg to about 200 MJ/kg, about 140 MJ/kg to about 160 MJ/kg, about 140 MJ/kg to about 180 MJ/kg, about 140 MJ/kg to about 200 MJ/kg, about 160 MJ/kg to about 180 MJ/kg, about 160 MJ/kg to about 200 MJ/kg, or about 180 MJ/kg to about 200 MJ/kg.

A modular cannabis production process may incorporate a step comprising converting one or more residual cannabis-derived biomass components into biochar. Biochar may be formed by the heating of biomass to a conversion temperature under anaerobic or low-oxygen conditions. Biochar may be formed in a batch process or in a continuous production process. Biochar may be formed in ovens, kilns, or continuous-feed reactors. A biochar conversion process may entail the partial or complete release of volatile matter from a biomass component. Biochar may be produced at a temperature of at least about 300° C., 325° C., 350° C., 375° C., 400° C., 425° C., 450° C., 475° C., 500° C., 525° C., 550° C., 575° C., or at least about 600° C. Biochar may be produced at a temperature no greater than about 600° C., 575° C., 550° C., 525° C., 500° C., 475° C., 450° C., 425° C., 400° C., 375° C., 350° C., 325° C., or no greater than about 300° C. Biochar may be produced in a temperature range between about 300° C. and about 350° C., 300° C. and about 400° C., 300° C. and about 450° C., 300° C. and about 500° C., 300° C. and about 550° C., 300° C. and about 600° C., 350° C. and about 400° C., 350° C. and about 450° C., 350° C. and about 500° C., 350° C. and about 550° C., 350° C. and about 600° C., 400° C. and about 450° C., 400° C. and about 500° C., 400° C. and about 550° C., 400° C. and about 600° C., 450° C. and about 500° C., 450° C. and about 550° C., 450° C. and about 600° C., 500° C. and about 550° C., 500° C. and about 600° C., or about 550° C. and about 600° C. The heating rate of a biochar conversion process may be about 0.1° C./second, 1° C./second, 10° C./second, 50° C./second, 100° C./second, 200° C./second, 300° C./second, 400° C./second, 500° C./second, 600° C./second, 700° C./second, 800° C./second, 900° C./second, or about 1000° C./second. The heating rate of the pyrolysis zone may be no less than about 0.1° C./second, 1° C./second, 10° C./second, 50° C./second, 100° C./second, 200° C./second, 300° C./second, 400° C./second, 500° C./second, 600° C./second, 700° C./second, 800° C./second, 900° C./second, or less than about 1000° C./second. The heating rate of the pyrolysis zone may be no greater than about 1000° C./second, 900° C./second, 800° C./second, 700° C./second, 600° C./second, 500° C./second, 400° C./second, 300° C./second, 200° C./second, 100° C./second, 50° C./second, 10° C./second, 1° C./second, or no greater than about 0.1° C./second. A biochar conversion reactor may have a total residence time of at least about 1 second, 5 seconds, 10 seconds, 20 seconds, 30 seconds, 60 seconds, 5 minutes, 10 minutes, 20 minutes, 30 minutes, 40 minutes, 50 minutes, 60 minutes, 2 hours, 3 hours, 4 hours, 5 hours, or at least 6 hours.

A biochar conversion reactor may have a total residence time of no more than 6 hours, 5 hours, 4 hours, 3 hours, 2 hours, 60 minutes, 50 minutes, 40 minutes, 30 minutes, 20 minutes, 10 minutes, 5 minutes, 60 seconds, 30 seconds, 20 seconds, 10 seconds, 5 seconds, or no more than about 1 second. A biochar conversion reactor may have a residence time range from about 1 second to about 10 seconds, about 1 second to about 30 seconds, about 1 second to about 60 seconds, about 1 second to about 10 minutes, about 1 second to about 30 minutes, about 1 second to about 60 minutes, about 1 second to about 2 hours, about 1 second to about 4 hours, about 1 second to about 6 hours, about 10 seconds to about 30 seconds, about 10 seconds to about 60 seconds, about 10 seconds to about 10 minutes, about 10 seconds to about 30 minutes, about 10 seconds to about 60 minutes, about 10 seconds to about 2 hours, about 10 seconds to about 4 hours, about 10 seconds to about 6 hours, about 30 seconds to about 60 seconds, about 30 seconds to about 10 minutes, about 30 seconds to about 30 minutes, about 30 seconds to about 60 minutes, about 30 seconds to about 2 hours, about 30 seconds to about 4 hours, about 30 seconds to about 6 hours, about 60 seconds to about 10 minutes, about 60 seconds to about 30 minutes, about 60 seconds to about 60 minutes, about 60 seconds to about 2 hours, about 60 seconds to about 4 hours, about 60 seconds to about 6 hours, about 10 minutes to about 30 minutes, about 10 minutes to about 60 minutes, about 10 minutes to about 2 hours, about 10 minutes to about 4 hours, about 10 minutes to about 6 hours, about 30 minutes to about 60 minutes, about 30 minutes to about 2 hours, about 30 minutes to about 4 hours, about 30 minutes to about 6 hours, about 60 minutes to about 2 hours, about 60 minutes to about 4 hours, about 60 minutes to about 6 hours, about 2 hours to about 4 hours, about 2 hours to about 6 hours, or about 4 hours to about 6 hours.

A biochar process may reduce the weight of a biomass feedstock due to the loss of volatile matter. A cannabis-derived biochar may have a volatile matter release of no less than about 10%, 15%, 20%, 25%, 30%, 35%, 40%, 45%, 50%, 55%, 60%, 65%, 70%, 75%, 80%, 85%, or about 90% relative to the initial dry weight of the feedstock. A cannabis-derived biochar may have a volatile matter content of no greater than about 90%, 85%, 80%, 75%, 70%, 65%, 60%, 55%, 50%, 45%, 40%, 35%, 30%, 25%, 20%, 15%, or no greater than about 10% relative to the initial dry weight of the feedstock. Biochar may be created under low-oxygen conditions. A biochar conversion process may have an oxygen partial pressure of no more than 100 torr, 90 torr, 80 torr, 70 torr, 60 torr, 50 torr, 40 torr, 30 torr, 20 torr, 10 torr, 5 torr, 1 torr, 0.1 torr, or 0.01 torr.

Volatile matter from biochar conversion processes may be captured. Volatile matter may include light gases such as methane and ethane. The light gases may have value as a heating gas. Volatile gases may be flared and used in heat recovery processes to enhance the energy efficiency of a modular cannabis production process. Volatile liquids may also be captured. Volatile liquids may include condensable hydrocarbons, oils, tars, light waxes, and low molecular weight aromatics. Liquids may be separated and refined create cannabis-derived oils, liquid fuels, or other valuable chemical products.

A modular cannabis production process may incorporate a step comprising a gasification process. A gasification process may be batch-fed or continuous. A gasification process may co-gasify with other solid fuels or catalysts. A gasification process may require the use of various oxidants, including oxygen, steam, carbon dioxide, and carbon monoxide. A gasifier unit may utilize an updraft design, a downdraft design, a fixed-bed design, or a fluidized-bed design. A gasification process may comprise one or more gasifier units. A gasification process may comprise complementary unit operations such as heat exchangers, pumps, compressors, fans, boilers, and filters. A gasifier system may comprise at least a pyrolysis zone and a gasification zone. Pyrolysis may be physically separate from combustion, or both types of reactions may occur in the same reaction unit. Pyrolysis of cannabis biomass during gasification may be controlled by temperature, pressure, and heating rate.

The pyrolysis temperature of a gasifier may be about 250° C., 300° C., 325° C., 350° C., 375° C., 400° C., 425° C., 450° C., 475° C., 500° C., 525° C., 550° C., 575° C., 600° C., 625° C., 650° C., 675° C., or about 700° C. The pyrolysis temperature of a gasifier may be at least 250° C., 300° C., 325° C., 350° C., 375° C., 400° C., 425° C., 450° C., 475° C., 500° C., 525° C., 550° C., 575° C., 600° C., 625° C., 650° C., 675° C., or about 700° C. The pyrolysis temperature of a gasifier may be no more than about 700° C., 675° C., 650° C., 625° C., 600° C., 575° C., 550° C., 525° C., 500° C., 475° C., 450° C., 425° C., 400° C., 375° C., 350° C., 325° C., 300° C., or no more than about 250° C. The pyrolysis temperature may occur in a range from about 250° C. to about 300° C., about 250° C. to about 350° C., about 250° C. to about 400° C., about 250° C. to about 450° C., about 250° C. to about 500° C., about 250° C. to about 550° C., about 250° C. to about 600° C., about 250° C. to about 650° C., about 250° C. to about 700° C., about 300° C. to about 350° C., about 300° C. to about 400° C., about 300° C. to about 450° C., about 300° C. to about 500° C., about 300° C. to about 550° C., about 300° C. to about 600° C., about 300° C. to about 650° C., about 300° C. to about 700° C., about 350° C. to about 400° C., about 350° C. to about 450° C., about 350° C. to about 500° C., about 350° C. to about 550° C., about 350° C. to about 600° C., about 350° C. to about 650° C., about 350° C. to about 700° C., about 400° C. to about 450° C., about 400° C. to about 500° C., about 400° C. to about 550° C., about 400° C. to about 600° C., about 400° C. to about 650° C., about 400° C. to about 700° C., about 450° C. to about 500° C., about 450° C. to about 550° C., about 450° C. to about 600° C., about 450° C. to about 650° C., about 450° C. to about 700° C., about 500° C. to about 550° C., about 500° C. to about 600° C., about 500° C. to about 650° C., about 500° C. to about 700° C., about 550° C. to about 600° C., about 550° C. to about 650° C., about 550° C. to about 700° C., about 600° C. to about 650° C., about 600° C. to about 700° C., or about 650° C. to about 700° C. The heating rate of the pyrolysis zone may be about 0.1° C./second, 1° C./second, 10° C./second, 50° C./second, 100° C./second, 200° C./second, 300° C./second, 400° C./second, 500° C./second, 600° C./second, 700° C./second, 800° C./second, 900° C./second, or about 1000° C./second. The heating rate of the pyrolysis zone may be no less than about 0.1° C./second, 1° C./second, 10° C./second, 50° C./second, 100° C./second, 200° C./second, 300° C./second, 400° C./second, 500° C./second, 600° C./second, 700° C./second, 800° C./second, 900° C./second, or less than about 1000° C./second. The heating rate of the pyrolysis zone may be no greater than about 1000° C./second, 900° C./second, 800° C./second, 700° C./second, 600° C./second, 500° C./second, 400° C./second, 300° C./second, 200° C./second, 100° C./second, 50° C./second, 10° C./second, 1° C./second, or no greater than about 0.1° C./second.

The pressure of the pyrolysis zone of a gasifier may be about 10 torr, 100 torr, 150 torr, 200 torr, 250 torr, 300 torr, 350 torr, 400 torr, 450 torr, 500 torr, 550 torr, 600 torr, 650 torr, 700 torr, 750 torr, 800 torr, 900 torr, 1000 torr, 1100 torr, 1200 torr, 1300 torr, 1400 torr, 1500 torr, 2000 torr, 2250 torr, 2500 torr, 3000 torr, 4000 torr, 5000 torr, 6000 torr, 7000 torr, or about 7500 torr. The pressure of the pyrolysis zone may be no less than about 10 torr, 100 torr, 150 torr, 200 torr, 250 torr, 300 torr, 350 torr, 400 torr, 450 torr, 500 torr, 550 torr, 600 torr, 650 torr, 700 torr, 750 torr, 800 torr, 900 torr, 1000 torr, 1100 torr, 1200 torr, 1300 torr, 1400 torr, 1500 torr, 2000 torr, 2250 torr, 2500 torr, 3000 torr, 4000 torr, 5000 torr, 6000 torr, 7000 torr, or no less than about 7500 torr. The pressure of the pyrolysis zone may be no greater than about 7500 torr, 6000 torr, 5000 torr, 4000 torr, 3000 torr, 2500 torr, 2250 torr, 2000 torr, 1500 torr, 1400 torr, 1300 torr, 1200 torr, 1100 torr, 1000 torr, 900 torr, 800 torr, 750 torr, 700 torr, 650 torr, 600 torr, 550 torr, 500 torr, 450 torr, 400 torr, 350 torr, 300 torr, 250 torr, 200 torr, 150 torr, 100 torr, or no greater than about 10 torr.

A biomass gasifier may comprise a gasification zone with an independent thermal characteristic from the pyrolysis zone. The gasification zone may comprise one or more ports for the flow of combustion gases such as steam, CO₂, and O₂. The gasification zone may be controlled for temperature, heating rate and pressure. The gasification temperature of a gasifier may be about 700° C., 750° C., 800° C., 850° C., 900° C., 950° C., 1000° C., 1050° C., 1100° C., 350° C., 1200° C., 1250° C., 1300° C., 1350° C., 1400° C., 1450° C., or about 1500° C. The gasification temperature of a gasifier may be at least about 700° C., 750° C., 800° C., 850° C., 900° C., 950° C., 1000° C., 1050° C., 1100° C., 350° C., 1200° C., 1250° C., 1300° C., 1350° C., 1400° C., 1450° C., or at least about 1500° C. The gasification temperature of a gasifier may be no more than about 1500° C., 1450° C., 1400° C., 1350° C., 1300° C., 1250° C., 1200° C., 350° C., 1100° C., 1050° C., 1000° C., 950° C., 900° C., 850° C., 800° C., 750° C., or no more than about 700° C. The gasification zone of a gasifier may have a temperature in a range from about 700° C. to about 800° C., about 700° C. to about 900° C., about 700° C. to about 1000° C., about 700° C. to about 1100° C., about 700° C. to about 1200° C., about 700° C. to about 1300° C., about 700° C. to about 1400° C., about 700° C. to about 1500° C., about 800° C. to about 900° C., about 800° C. to about 1000° C., about 800° C. to about 1100° C., about 800° C. to about 1200° C., about 800° C. to about 1300° C., about 800° C. to about 1400° C., about 800° C. to about 1500° C., about 900° C. to about 1000° C., about 900° C. to about 1100° C., about 900° C. to about 1200° C., about 900° C. to about 1300° C., about 900° C. to about 1400° C., about 900° C. to about 1500° C., about 1000° C. to about 1100° C., about 1000° C. to about 1200° C., about 1000° C. to about 1300° C., about 1000° C. to about 1400° C., about 1000° C. to about 1500° C., about 1100° C. to about 1200° C., about 1100° C. to about 1300° C., about 1100° C. to about 1400° C., about 1100° C. to about 1500° C., about 1200° C. to about 1300° C., about 1200° C. to about 1400° C., about 1200° C. to about 1500° C., about 1300° C. to about 1400° C., about 1300° C. to about 1500° C., or from about 1400° C. to about 1500° C. The heating rate of the gasification zone may be no less than about 0.1° C./second, 1° C./second, 10° C./second, 50° C./second, 100° C./second, 200° C./second, 300° C./second, 400° C./second, 500° C./second, 600° C./second, 700° C./second, 800° C./second, 900° C./second, or less than about 1000° C./second. The heating rate of the gasification zone may be no greater than about 1000° C./second, 900° C./second, 800° C./second, 700° C./second, 600° C./second, 500° C./second, 400° C./second, 300° C./second, 200° C./second, 100° C./second, 50° C./second, 10° C./second, 1° C./second, or no greater than about 0.1° C./second.

The pressure of the gasification zone of a gasifier may be about 10 torr, 100 torr, 150 torr, 200 torr, 250 torr, 300 torr, 350 torr, 400 torr, 450 torr, 500 torr, 550 torr, 600 torr, 650 torr, 700 torr, 750 torr, 800 torr, 900 torr, 1000 torr, 1100 torr, 1200 torr, 1300 torr, 1400 torr, 1500 torr, 2000 torr, 2250 torr, 2500 torr, 3000 torr, 4000 torr, 5000 torr, 6000 torr, 7000 torr, or about 7500 torr. The pressure of the gasification zone may be no less than about 10 torr, 100 torr, 150 torr, 200 torr, 250 torr, 300 torr, 350 torr, 400 torr, 450 torr, 500 torr, 550 torr, 600 torr, 650 torr, 700 torr, 750 torr, 800 torr, 900 torr, 1000 torr, 1100 torr, 1200 torr, 1300 torr, 1400 torr, 1500 torr, 2000 torr, 2250 torr, 2500 torr, 3000 torr, 4000 torr, 5000 torr, 6000 torr, 7000 torr, or no less than about 7500 torr. The pressure of the gasification zone may be no greater than about 7500 torr, 6000 torr, 5000 torr, 4000 torr, 3000 torr, 2500 torr, 2250 torr, 2000 torr, 1500 torr, 1400 torr, 1300 torr, 1200 torr, 1100 torr, 1000 torr, 900 torr, 800 torr, 750 torr, 700 torr, 650 torr, 600 torr, 550 torr, 500 torr, 450 torr, 400 torr, 350 torr, 300 torr, 250 torr, 200 torr, 150 torr, 100 torr, or no greater than about 10 torr.

The gasifier fuel may be gasified until all volatile matter is consumed. A gasifier fuel may be partially gasified in a single pass through a gasification reactor. A gasifier fuel may be recycled through a gasifier or processed through a successive gasifier to enhance the extent of gasification. A gasifier fuel may release at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or about 99.9% of its volatile matter during a gasification reaction. A gasifier fuel may release no more than about 99.9%, 99%, 98%, 97%, 96%, 95%, 90%, 85%, 80%, 75%, 70%, 65%, 60%, 55%, 50%, 45%, 40%, 35%, 30%, 25%, 20%, 15% or no more than about 10% of its volatile matter during a gasification reaction. A gasifier fuel may have a total residence time of at least about 1 second, 5 seconds, 10 seconds, 20 seconds, 30 seconds, 60 seconds, 5 minutes, 10 minutes, 20 minutes, 30 minutes, 40 minutes, 50 minutes, 60 minutes, 2 hours, 3 hours, 4 hours, 5 hours, 6 hours, or at least about 12 hours. A gasifier fuel may have a total residence time of no more than 12 hours, 6 hours, 5 hours, 4 hours, 3 hours, 2 hours, 60 minutes, 50 minutes, 40 minutes, 30 minutes, 20 minutes, 10 minutes, 5 minutes, 60 seconds, 30 seconds, 20 seconds, 10 seconds, 5 seconds, or no more than about 1 second. A gasifier fuel may have a residence time range from about 1 second to about 10 seconds, about 1 second to about 30 seconds, about 1 second to about 60 seconds, about 1 second to about 10 minutes, about 1 second to about 30 minutes, about 1 second to about 60 minutes, about 1 second to about 2 hours, about 1 second to about 4 hours, about 1 second to about 6 hours, about 1 second to about 12 hours, about 10 seconds to about 30 seconds, about 10 seconds to about 60 seconds, about 10 seconds to about 10 minutes, about 10 seconds to about 30 minutes, about 10 seconds to about 60 minutes, about 10 seconds to about 2 hours, about 10 seconds to about 4 hours, about 10 seconds to about 6 hours, about 10 seconds to about 12 hours, about 30 seconds to about 60 seconds, about 30 seconds to about 10 minutes, about 30 seconds to about 30 minutes, about 30 seconds to about 60 minutes, about 30 seconds to about 2 hours, about 30 seconds to about 4 hours, about 30 seconds to about 6 hours, about 30 seconds to about 12 hours, about 60 seconds to about 10 minutes, about 60 seconds to about 30 minutes, about 60 seconds to about 60 minutes, about 60 seconds to about 2 hours, about 60 seconds to about 4 hours, about 60 seconds to about 6 hours, about 60 seconds to about 12 hours, about 10 minutes to about 30 minutes, about 10 minutes to about 60 minutes, about 10 minutes to about 2 hours, about 10 minutes to about 4 hours, about 10 minutes to about 6 hours, about 10 minutes to about 12 hours, about 30 minutes to about 60 minutes, about 30 minutes to about 2 hours, about 30 minutes to about 4 hours, about 30 minutes to about 6 hours, about 30 minutes to about 12 hours, about 60 minutes to about 2 hours, about 60 minutes to about 4 hours, about 60 minutes to about 6 hours, about 60 minutes to about 12 hours, about 2 hours to about 4 hours, about 2 hours to about 6 hours, about 2 hours to about 12 hours, about 4 hours to about 6 hours, about 4 hours to about 12 hours, or about 6 hours to about 12 hours.

A gasification process utilizing cannabis components as a fuel may generate a producer gas or a synthesis gas (syngas). The producer gas may comprise a mixture of hydrogen, carbon monoxide, carbon dioxide, water, and other light hydrocarbons such as methane. The producer gas may be purified via one or more separation units, such as distillation columns, membrane separations, gas absorbers, or gas strippers. Producer gas may be directly combusted to capture energy via heat recovery. Producer gas may be combusted in a fuel cell to directly generate electrical energy. Producer gas may be converted into liquid hydrocarbons via a Fischer-Tropsch reaction or other method of chemical conversion. A producer gas produced via the gasification of cannabis biomass components may be characterized by a higher heating value. A biomass-derived syngas may have a higher heating value of at least about 10 MJ/kg, 20 MJ/kg, 30 MJ/kg, 40 MJ/kg, 50 MJ/kg, 60 MJ/kg, 70 MJ/kg, 80 MJ/kg, 90 MJ/kg, 100 MJ/kg, 120 MJ/kg, 140 MJ/kg, 160 MJ/kg, 180 MJ/kg, or at least about 200 MJ/kg. A biomass-derived syngas may have a higher heating value of no greater than about 200 MJ/kg, 180 MJ/kg, 160 MJ/kg, 140 MJ/kg, 120 MJ/kg, 100 MJ/kg, 90 MJ/kg, 80 MJ/kg, 70 MJ/kg, 60 MJ/kg, 50 MJ/kg, 40 MJ/kg, 30 MJ/kg, 20 MJ/kg, or no greater than about 10 MJ/kg. A biomass-derived syngas may have a higher heating value in a range from about 10 MJ/kg to about 20 MJ/kg, about 10 MJ/kg to about 40 MJ/kg, about 10 MJ/kg to about 60 MJ/kg, about 10 MJ/kg to about 80 MJ/kg, about 10 MJ/kg to about 100 MJ/kg, about 10 MJ/kg to about 120 MJ/kg, about 10 MJ/kg to about 140 MJ/kg, about 10 MJ/kg to about 160 MJ/kg, about 10 MJ/kg to about 180 MJ/kg, about 10 MJ/kg to about 200 MJ/kg, about 20 MJ/kg to about 40 MJ/kg, about 20 MJ/kg to about 60 MJ/kg, about 20 MJ/kg to about 80 MJ/kg, about 20 MJ/kg to about 100 MJ/kg, about 20 MJ/kg to about 120 MJ/kg, about 20 MJ/kg to about 140 MJ/kg, about 20 MJ/kg to about 160 MJ/kg, about 20 MJ/kg to about 180 MJ/kg, about 20 MJ/kg to about 200 MJ/kg, about 40 MJ/kg to about 60 MJ/kg, about 40 MJ/kg to about 80 MJ/kg, about 40 MJ/kg to about 100 MJ/kg, about 40 MJ/kg to about 120 MJ/kg, about 40 MJ/kg to about 140 MJ/kg, about 40 MJ/kg to about 160 MJ/kg, about 40 MJ/kg to about 180 MJ/kg, about 40 MJ/kg to about 200 MJ/kg, about 60 MJ/kg to about 80 MJ/kg, about 60 MJ/kg to about 100 MJ/kg, about 60 MJ/kg to about 120 MJ/kg, about 60 MJ/kg to about 140 MJ/kg, about 60 MJ/kg to about 160 MJ/kg, about 60 MJ/kg to about 180 MJ/kg, about 60 MJ/kg to about 200 MJ/kg, about 80 MJ/kg to about 100 MJ/kg, about 80 MJ/kg to about 120 MJ/kg, about 80 MJ/kg to about 140 MJ/kg, about 80 MJ/kg to about 160 MJ/kg, about 80 MJ/kg to about 180 MJ/kg, about 80 MJ/kg to about 200 MJ/kg, about 100 MJ/kg to about 120 MJ/kg, about 100 MJ/kg to about 140 MJ/kg, about 100 MJ/kg to about 160 MJ/kg, about 100 MJ/kg to about 180 MJ/kg, about 100 MJ/kg to about 200 MJ/kg, about 120 MJ/kg to about 140 MJ/kg, about 120 MJ/kg to about 160 MJ/kg, about 120 MJ/kg to about 180 MJ/kg, about 120 MJ/kg to about 200 MJ/kg, about 140 MJ/kg to about 160 MJ/kg, about 140 MJ/kg to about 180 MJ/kg, about 140 MJ/kg to about 200 MJ/kg, about 160 MJ/kg to about 180 MJ/kg, about 160 MJ/kg to about 200 MJ/kg, or about 180 MJ/kg to about 200 MJ/kg.

A modular cannabis production process may convert residual cannabis biomass into pellets for use as a biomass fuel. Pelletization may utilize any biomass component including barks, stems, roots, leaves, hurd, flowering bodies, and any residual materials from other processes such as cannabinoid extraction. Biomass may be chipped, shredded, milled, or otherwise rendered to make it of a sufficient texture and quality for pelletization. Pelletization may occur in a batch process or a continuous process. A continuous process may utilize subsidiary components such as oven dryers, screw augers, hoppers, and extruders. An extrusion process for pellet production may involve the compression of biomass material in a mold and dye configuration. Pelletized biomass may have an optimal moisture content during the pelletization process. Pelletized biomass may have a moisture content of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or about 30% on a weight basis during the pelletization process. A biomass pelletization process may require a minimum moisture content of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or about 30% on a weight basis. A biomass pelletization process may require the biomass moisture content to be no more than about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or no more than about 1% on a weight basis. Biomass may be pelletized with a moisture content in a range from about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30% on a weight basis.

Fuel pellets produced from residual cannabis biomass may be sized to a particular application. Fuel pellets may be produced to a particular size standard or for an intended market. Fuel pellets can be altered for a particular diameter, length, and density. A cannabis-derived fuel pellet may have a diameter of about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or about 30 mm. A cannabis-derived fuel pellet may have a diameter of no less than about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or no less than about 30 mm. A cannabis-derived fuel pellet may have a diameter of no more than about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, or no more than about 1 mm. A cannabis-derived biomass pellet may have a length of about 5 mm, 10 mm, 15 mm, 20 mm, 25 mm, 30 mm, 35 mm, 40 mm, 45 mm, or about 50 mm. A cannabis-derived biomass pellet may have a length of no less than about 5 mm, 10 mm, 15 mm, 20 mm, 25 mm, 30 mm, 35 mm, 40 mm, 45 mm, or about 50 mm. A cannabis-derived biomass pellet may have a length of no more than about 50 mm, 45 mm, 40 mm, 35 mm, 30 mm, 25 mm, 20 mm, 15 mm, 10 mm, or no more than about 5 mm. A cannabis-derived pellet may have a length in a range from about 5 mm to 10 mm, 5 mm to about 15 mm, about 5 mm to 20 mm, 5 mm to about 25 mm, about 5 mm to 30 mm, 5 mm to about 35 mm, about 5 mm to 40 mm, 5 mm to about 45 mm, about 5 mm to about 50 mm, 10 mm to about 15 mm, about 10 mm to 20 mm, 10 mm to about 25 mm, about 10 mm to 30 mm, 10 mm to about 35 mm, about 10 mm to 40 mm, 10 mm to about 45 mm, about 10 mm to about 50 mm, about 15 mm to 20 mm, 15 mm to about 25 mm, about 15 mm to 30 mm, 15 mm to about 35 mm, about 15 mm to 40 mm, 15 mm to about 45 mm, about 15 mm to about 50 mm, 20 mm to about 25 mm, about 20 mm to 30 mm, 20 mm to about 35 mm, about 20 mm to 40 mm, 20 mm to about 45 mm, about 20 mm to about 50 mm, about 25 mm to 30 mm, 25 mm to about 35 mm, about 25 mm to 40 mm, 25 mm to about 45 mm, about 25 mm to about 50 mm, 30 mm to about 35 mm, about 30 mm to 40 mm, 30 mm to about 45 mm, about 30 mm to about 50 mm, about 35 mm to 40 mm, 35 mm to about 45 mm, about 35 mm to about 50 mm, 40 mm to about 45 mm, about 40 mm to about 50 mm, or about 45 mm to about 50 mm.

A cannabis-derived pellet may have a density of no less than about 100 kilograms per cubic meter (kg/m³), 150 kg/m³, 200 kg/m³, 250 kg/m³, 300 kg/m³, 350 kg/m³, 400 kg/m³, 450 kg/m³, 500 kg/m³, 550 kg/m³, 600 kg/m³, 650 kg/m³, 700 kg/m³, 750 kg/m³, 800 kg/m³, 850 kg/m³, or no less than about 900 kg/m³. A cannabis-derived pellet may have a density of no more than about 900 kg/m³, 850 kg/m³, 800 kg/m³, 750 kg/m³, 700 kg/m³, 650 kg/m³, 600 kg/m³, 550 kg/m³, 500 kg/m³, 450 kg/m³, 400 kg/m³, 350 kg/m³, 300 kg/m³, 250 kg/m³, 200 kg/m³, 150 kg/m³, or no more than about 100 kg/m³. A cannabis-derived pellet may have a density in a range from about 100 kg/m³ to about 200 kg/m³, about 100 kg/m³ to about 300 kg/m³, about 100 kg/m³ to about 400 kg/m³, about 100 kg/m³ to about 500 kg/m³, about 100 kg/m³ to about 600 kg/m³, about 100 kg/m³ to about 700 kg/m³, about 100 kg/m³ to about 800 kg/m³, about 100 kg/m³ to about 900 kg/m³, about 200 kg/m³ to about 300 kg/m³, about 200 kg/m³ to about 400 kg/m³, about 200 kg/m³ to about 500 kg/m³, about 200 kg/m³ to about 600 kg/m³, about 200 kg/m³ to about 700 kg/m³, about 200 kg/m³ to about 800 kg/m³, about 200 kg/m³ to about 900 kg/m³, about 300 kg/m³ to about 400 kg/m³, about 300 kg/m³ to about 500 kg/m³, about 300 kg/m³ to about 600 kg/m³, about 300 kg/m³ to about 700 kg/m³, about 300 kg/m³ to about 800 kg/m³, about 300 kg/m³ to about 900 kg/m³, about 400 kg/m³ to about 500 kg/m³, about 400 kg/m³ to about 600 kg/m³, about 400 kg/m³ to about 700 kg/m³, about 400 kg/m³ to about 800 kg/m³, about 400 kg/m³ to about 900 kg/m³, about 500 kg/m³ to about 600 kg/m³, about 500 kg/m³ to about 700 kg/m³, about 500 kg/m³ to about 800 kg/m³, about 500 kg/m³ to about 900 kg/m³, about 600 kg/m³ to about 700 kg/m³, about 600 kg/m³ to about 800 kg/m³, about 600 kg/m³ to about 900 kg/m³, about 700 kg/m³ to about 800 kg/m³, about 700 kg/m³ to about 900 kg/m³, about 800 kg/m³ to about 900 kg/m³.

Biomass derived from cannabis plants may be chemically converted into other useful materials. In some instances, a component, material, or chemical substance derived from cannabis may be converted in to a plastic or polymeric material. In some instances, a cannabis plant component, such as seed hulls, may be processed into a surfactant based upon materials derived from the seed hulls. In some instances, biomass, such as bast fiber material, can be used as precursors to manufacture components of capacitors such as, for example, graphene-like carbon nanosheet structures (e.g., carbon sheets having dimensions from 1 nanometer to at most 1000 nanometers or 500 nanometers) using conventional processes, such as hydrothermal synthesis. Such precursors may be formed in the form of sheets, tubes, or rolls, for example. For example, bast fiber can first undergo hydrothermal carbonization to break up an initially yarn like structure of the bast fiber into smaller pieces. The hydrothermal synthesis process can yield high oxygen content (e.g., oxygen-containing functional groups), making the yield susceptible to a subsequent activation process using activating reagents such as potassium hydroxide (KOH). After the hydrothermal process, the bast fiber can then be activated with, e.g., KOH, to penetrate the bast fiber and generate carbon nanosheets. The activation temperature can be at least about 600 degrees Celsius (° C.), 650° C., 700° C., 705° C., 710° C., 715° C., 720° C., 725° C., 730° C., 735° C., 740° C., 745° C., 750° C., 755° C., 760° C., 765° C., 770° C., 775° C., 780° C., 785° C., 790° C., 795° C., 800° C. or higher. As an alternative the activation temperature can be less than or equal to about 800° C., 790° C., 780° C., 770° C., 760° C., 750° C., 740° C., 730° C., 720° C., 710° C., 700° C., 650° C., 600° C. or lower.

A modular cannabis production process may include one or more units that perform cannabinoid extraction from various Cannabis-derived biomass components. Cannabinoid extractions may comprise any method that selectively removes the cannabinoid content of cannabis biomass while retaining as much of the solid structure of the biomass as possible. Cannabinoid extractions may include solvent extractions (e.g. butane extraction),supercritical CO₂ extractions, and pressed extractions. Cannabinoid extractions may be performed in batch or continuous processes. Cannabinoid extractions may require one or more extraction devices. Cannabinoid extractions may include ancillary components such as distillation devices, liquid-liquid separation units, chromatography units, pumps, compressors, chillers, and heaters. Cannabinoid extraction processes may be carefully controlled for temperature, pressure, and residence time of the biomass material in the extraction device.

Solvent extractions may be performed on various cannabis-derived biomass components, including seeds, trichomes, leaves, stems, and buds. Solvents may comprise ethanol, propane, and butane. Solvent extraction may be performed at a broad range of temperatures depending upon the desired outcome of the extraction process. Solvent extraction may be performed at a temperature of about −80° C., −70° C., −60° C., −50° C., −40° C., −30° C., −20° C., −10° C., 0° C., 10° C., 20° C., 30° C., 40° C., 50° C., 60° C., 70° C., or about 80° C. Solvent extraction may be performed at a temperature that is no less than about −80° C., −70° C., −60° C., −50° C., −40° C., −30° C., −20° C., −10° C., 0° C., 10° C., 20° C., 30° C., 40° C., 50° C., 60° C., 70° C., or no less than about 80° C. Solvent extraction may be performed at a temperature that is no greater than about 80° C., 70° C., 60° C., 50° C., 40° C., 30° C., 20° C., 10° C., 0° C., −10° C., −20° C., −30° C., −40° C., −50° C., −60° C., −70° C., or no greater than about −80° C. Solvent extraction may occur in a temperature range from about −80° C. to about −60° C., about −80° C. to about −40° C., about −80° C. to about −20° C., about −80° C. to about 0° C., about −80° C. to about 20° C., about −80° C. to about 40° C., about −80° C. to about 60° C., about −80° C. to about 80° C., about −60° C. to about −40° C., about −60° C. to about −20° C., about −60° C. to about 0° C., about −60° C. to about 20° C., about −60° C. to about 40° C., about −60° C. to about 60° C., about −60° C. to about 80° C., about −40° C. to about −20° C., about −40° C. to about 0° C., about −40° C. to about 20° C., about −40° C. to about 40° C., about −40° C. to about 60° C., about −40° C. to about 80° C., about −20° C. to about 0° C., about −20° C. to about 20° C., about −20° C. to about 40° C., about −20° C. to about 60° C., about −20° C. to about 80° C., about 0° C. to about 20° C., about 0° C. to about 40° C., about 0° C. to about 60° C., about 0° C. to about 80° C., about 20° C. to about 40° C., about 20° C. to about 60° C., about 20° C. to about 80° C., about 40° C. to about 60° C., about 40° C. to about 80° C., or about 60° C. to about 80° C.

Solvent extraction of cannabinoids may be performed at about atmospheric pressure. Solvent extraction of cannabinoids may be performed at a pressure above atmospheric pressure. A solvent extraction may be performed at a pressure of about 2 bar, 3 bar, 4 bar, 5 bar, 10 bar, 20 bar, 40 bar, 60 bar, 80 bar, 100 bar, 150 bar, or about 200 bar. A solvent extraction of cannabinoids may be performed at a pressure of at least 2 bar, 3 bar, 4 bar, 5 bar, 10 bar, 20 bar, 40 bar, 60 bar, 80 bar, 100 bar, 150 bar, or at least about 200 bar. A solvent extraction of cannabinoids may be performed at a pressure of no greater than about 200 bar, 150 bar, 100 bar, 80 bar, 60 bar, 40 bar, 20 bar, 10 bar, 5 bar, 4 bar, 3 bar, or no greater than about 2 bar. A solvent extraction may have a variable pressure with a pressure range from about 1 bar to about 2 bar, 1 bar to about 5 bar, 1 bar to about 10 bar, 1 bar to about 20 bar, 1 bar to about 40 bar, 1 bar to about 60 bar, 1 bar to about 80 bar, 1 bar to about 100 bar, 1 bar to about 150 bar, 1 bar to about 200 bar, 2 bar to about 5 bar, 2 bar to about 10 bar, 2 bar to about 20 bar, 2 bar to about 40 bar, 2 bar to about 60 bar, 2 bar to about 80 bar, 2 bar to about 100 bar, 2 bar to about 150 bar, 2 bar to about 200 bar, 5 bar to about 10 bar, 5 bar to about 20 bar, 5 bar to about 40 bar, 5 bar to about 60 bar, 5 bar to about 80 bar, 5 bar to about 100 bar, 5 bar to about 150 bar, 5 bar to about 200 bar, 10 bar to about 20 bar, 10 bar to about 40 bar, 10 bar to about 60 bar, 10 bar to about 80 bar, 10 bar to about 100 bar, 10 bar to about 150 bar, 10 bar to about 200 bar, 20 bar to about 40 bar, 20 bar to about 60 bar, 20 bar to about 80 bar, 20 bar to about 100 bar, 20 bar to about 150 bar, 20 bar to about 200 bar, 40 bar to about 60 bar, 40 bar to about 80 bar, 40 bar to about 100 bar, 40 bar to about 150 bar, 40 bar to about 200 bar, 60 bar to about 80 bar, 60 bar to about 100 bar, 60 bar to about 150 bar, 60 bar to about 200 bar, 80 bar to about 100 bar, 80 bar to about 150 bar, 80 bar to about 200 bar, 100 bar to about 150 bar, 100 bar to about 200 bar, or about 150 bar to about 200 bar. A solvent extraction process for cannabinoids from cannabis biomass may occur for a residence time of about 1 min, 5 mins, 10 mins, 30 mins, 60 mins, 2 hours, 4 hours, 6 hours, or about 12 hours. A solvent extraction process for cannabinoids from cannabis biomass may occur for a residence time of at least about 1 min, 5 mins, 10 mins, 30 mins, 60 mins, 2 hours, 4 hours, 6 hours, or at least about 12 hours. A solvent extraction process for cannabinoids from cannabis biomass may occur for a residence time of no greater than about 12 hours, 6 hours, 4 hours, 2 hours, 60 mins, 30 mins, 10 mins, 5 mins or 1 min.

Supercritical CO₂ extractions may be performed on various cannabis-derived biomass components, including seeds, trichomes, leaves, stems, and buds. Additional solvents, such as ethanol, may be employed depending upon the desired outcome of the extraction. Supercritical CO₂ extraction may be performed at a broad range of temperatures depending upon the desired outcome of the extraction process. Supercritical CO₂ extraction will always occur at a temperature and pressure above the critical point of CO₂, namely 31° C. and 74 bar. Supercritical CO₂ extraction may be performed at a temperature of about 31° C., 40° C., 50° C., 60° C., 70° C., 80° C., 90° C., 100° C., 110° C., 120° C., 130° C., 140° C., or about 150° C. Supercritical CO₂ extraction may be performed at a temperature that is no less than about 31° C., 40° C., 50° C., 60° C., 70° C., 80° C., 90° C., 100° C., 110° C., 120° C., 130° C., 140° C., or about 150° C. Supercritical CO₂ extraction may be performed at a temperature that is no greater than about 150° C., 140° C., 130° C., 120° C., 110° C., 100° C., 90° C., 80° C., 70° C., 60° C., 50° C., 40° C., or no greater than about 31° C. Supercritical CO₂ extraction may occur in a temperature range from about 31° C. to about 50° C., about 31° C. to about 70° C., about 31° C. to about 90° C., about 31° C. to about 110° C., about 31° C. to about 130° C., about 31° C. to about 150° C., about 50° C. to about 70° C., about 50° C. to about 90° C., about 50° C. to about 110° C., about 50° C. to about 130° C., about 50° C. to about 150° C., about 70° C. to about 90° C., about 70° C. to about 110° C., about 70° C. to about 130° C., about 70° C. to about 150° C., about 90° C. to about 110° C., about 90° C. to about 130° C., about 90° C. to about 150° C., about 110° C. to about 130° C., about 110° C. to about 150° C., or about 130° C. to about 150° C.

Supercritical CO₂ extraction of cannabinoids may be performed at a pressure above atmospheric pressure. A supercritical CO₂ extraction may be performed at a pressure of about 74 bar, 80 bar, 100 bar, 120 bar, 140 bar, 160 bar, 180 bar, 200 bar, 250 bar, 300 bar, 350 bar, 400 bar, 450 bar, or about 500 bar. A supercritical CO₂ extraction of cannabinoids may be performed at a pressure of at least about 74 bar, 80 bar, 100 bar, 120 bar, 140 bar, 160 bar, 180 bar, 200 bar, 250 bar, 300 bar, 350 bar, 400 bar, 450 bar, or about 500 bar. A supercritical CO₂ extraction of cannabinoids may be performed at a pressure of no greater than about 500 bar, 450 bar, 400 bar, 350 bar, 300 bar, 250 bar, 200 bar, 180 bar, 160 bar, 140 bar, 120 bar, 100 bar, 80 bar, or no greater than about 74 bar. A supercritical CO₂ extraction may have a variable pressure with a pressure range from about 74 bar to about 80 bar, about 74 bar to about 100 bar, about 74 bar to about 120 bar, about 74 bar to about 80 bar, about 74 bar to about 140 bar, about 74 bar to about 160 bar, about 74 bar to about 180 bar, about 74 bar to about 200 bar, about 74 bar to about 300 bar, about 74 bar to about 400 bar, about 74 bar to about 500 bar, about 80 bar to about 100 bar, about 80 bar to about 120 bar, about 80 bar to about 80 bar, about 80 bar to about 140 bar, about 80 bar to about 160 bar, about 80 bar to about 180 bar, about 80 bar to about 200 bar, about 80 bar to about 300 bar, about 80 bar to about 400 bar, about 80 bar to about 500 bar, about 100 bar to about 120 bar, about 100 bar to about 80 bar, about 100 bar to about 140 bar, about 100 bar to about 160 bar, about 100 bar to about 180 bar, about 100 bar to about 200 bar, about 100 bar to about 300 bar, about 100 bar to about 400 bar, about 100 bar to about 500 bar, about 120 bar to about 140 bar, about 120 bar to about 160 bar, about 120 bar to about 180 bar, about 120 bar to about 200 bar, about 120 bar to about 300 bar, about 120 bar to about 400 bar, about 120 bar to about 500 bar, about 140 bar to about 160 bar, about 140 bar to about 180 bar, about 140 bar to about 200 bar, about 140 bar to about 300 bar, about 140 bar to about 400 bar, about 140 bar to about 500 bar, about 160 bar to about 180 bar, about 160 bar to about 200 bar, about 160 bar to about 300 bar, about 160 bar to about 400 bar, about 160 bar to about 500 bar, about 180 bar to about 200 bar, about 180 bar to about 300 bar, about 180 bar to about 400 bar, about 180 bar to about 500 bar, about 200 bar to about 300 bar, about 200 bar to about 400 bar, about 200 bar to about 500 bar, about 300 bar to about 400 bar, about 300 bar to about 500 bar, or from about 400 bar to about 500 bar. A supercritical CO₂ extraction process for cannabinoids from cannabis biomass may occur for a residence time of about 1 min, 5 mins, 10 mins, 30 mins, 60 mins, 2 hours, 4 hours, 6 hours, or about 12 hours. A supercritical CO₂ extraction process for cannabinoids from cannabis biomass may occur for a residence time of at least about 1 min, 5 mins, 10 mins, 30 mins, 60 mins, 2 hours, 4 hours, 6 hours, or at least about 12 hours. A supercritical CO₂ extraction process for cannabinoids from cannabis biomass may occur for a residence time of no greater than about 12 hours, 6 hours, 4 hours, 2 hours, 60 mins, 30 mins, 10 mins, 5 mins or 1 min

A pressed extraction method may be utilized to extract cannabinoids from a cannabis plant material. A pressed extraction method may comprise the additional step of grinding or milling cannabis plant materials before pressing. A pressed extraction method may comprise a cold pressing method. Cold press extraction of cannabis plant materials may occur at a temperature of about 20° C. or less. Pressed extraction may occur in air or under a non-oxidizing atmosphere, such as nitrogen gas. A pressed extraction method may include the injection of a solvent or diluent to enhance the recovery of pressed liquids from the cannabis plant material. In some instances, cannabis seed hulls may be pressed to create an oil.

An extraction process may comprise an additional process, method, device, or component that allows the cannabinoid composition of the extracted oil to be altered. In some aspects, an alteration may comprise removing or diluting the concentration of THC relative to the concentration of CBD. In other aspects, an alteration may comprise converting THC to CBN or another cannabinoid. The additional process, method, device, or component may be employed until the concentration of THC drops to a prescribed level in the extracted oil. An extracted oil may have a THC concentration of less than about 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or less than about 3.0% on a weight basis. An extracted oil may have a THC concentration of about 0.01% to about 0.05%, about 0.01% to about 0.1%, about 0.01% to about 0.3%, about 0.01% to about 0.5%, about 0.01% to about 1.0%, about 0.01% to about 1.5%, about 0.01% to about 2.0%, about 0.01% to about 2.5%, about 0.01% to about 3.0%, about 0.05% to about 0.1%, about 0.05% to about 0.3%, about 0.05% to about 0.5%, about 0.05% to about 1.0%, about 0.05% to about 1.5%, about 0.05% to about 2.0%, about 0.05% to about 2.5%, about 0.05% to about 3.0%, about 0.1% to about 0.3%, about 0.1% to about 0.5%, about 0.1% to about 1.0%, about 0.1% to about 1.5%, about 0.1% to about 2.0%, about 0.1% to about 2.5%, about 0.1% to about 3.0%, about 0.3% to about 0.5%, about 0.3% to about 1.0%, about 0.3% to about 1.5%, about 0.3% to about 2.0%, about 0.3% to about 2.5%, about 0.3% to about 3.0%, about 0.5% to about 1.0%, about 0.5% to about 1.5%, about 0.5% to about 2.0%, about 0.5% to about 2.5%, about 0.5% to about 3.0%, about 1.0% to about 1.5%, about 1.0% to about 2.0%, about 1.0% to about 2.5%, about 1.0% to about 3.0%, about 1.5% to about 2.0%, about 1.5% to about 2.5%, about 1.5% to about 3.0%, about 2.0% to about 2.5%, about 2.0% to about 3.0%, or about 2.5% to about 3.0%.

In one aspect, an extraction processing unit may comprise a chamber for the dilution of THC from the extract oil. The dilution chamber may comprise a secondary solvent that preferentially solvates THC over CBD. The extraction unit may be stirred or agitated to increase contact time between the two solvents, enhancing the extraction of THC into the secondary solvent phase. In another aspect, an extraction processing unit may be operatively connected to a second separation unit comprising an adsorbent or chromatographic medium that preferentially binds THC. Extraction may be a continuous process where the extraction solution is fed to consecutive units. Extraction may be a recycling process where extract oils are iteratively passed between an extraction unit and a THC removal unit.

In another aspect, a UV light chamber may be coupled to an extraction processing unit. The UV light may irradiate the fluids within the extraction unit. The UV light may be configured to irradiate an exit port or a standalone unit that recycles fluids back to the extraction unit. The UV light may be of a sufficient wavelength, intensity, and residence time to convert some or all of the THC to CBN or perform other photochemical conversions. The wavelength of the light may be chosen to optimize a particular photochemical conversion. The UV light may have a wavelength of about 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, or about 400 nm. The UV light may have a range from about 200 nm to about 220 nm, about 200 nm to about 240 nm, about 200 nm to about 260 nm, about 200 nm to about 280 nm, about 200 nm to about 300 nm, about 200 nm to about 320 nm, about 200 nm to about 340 nm, about 200 nm to about 360 nm, about 200 nm to about 380 nm, about 200 nm to about 400 nm, about 220 nm to about 240 nm, about 220 nm to about 260 nm, about 220 nm to about 280 nm, about 220 nm to about 300 nm, about 220 nm to about 320 nm, about 220 nm to about 340 nm, about 220 nm to about 360 nm, about 220 nm to about 380 nm, about 220 nm to about 400 nm, about 240 nm to about 260 nm, about 240 nm to about 280 nm, about 240 nm to about 300 nm, about 240 nm to about 320 nm, about 240 nm to about 340 nm, about 240 nm to about 360 nm, about 240 nm to about 380 nm, about 240 nm to about 400 nm, about 260 nm to about 280 nm, about 260 nm to about 300 nm, about 260 nm to about 320 nm, about 260 nm to about 340 nm, about 260 nm to about 360 nm, about 260 nm to about 380 nm, about 260 nm to about 400 nm, about 280 nm to about 300 nm, about 280 nm to about 320 nm, about 280 nm to about 340 nm, about 280 nm to about 360 nm, about 280 nm to about 380 nm, about 280 nm to about 400 nm, about 300 nm to about 320 nm, about 300 nm to about 340 nm, about 300 nm to about 360 nm, about 300 nm to about 380 nm, about 300 nm to about 400 nm, about 320 nm to about 340 nm, about 320 nm to about 360 nm, about 320 nm to about 380 nm, about 320 nm to about 400 nm, about 340 nm to about 360 nm, about 340 nm to about 380 nm, about 340 nm to about 400 nm, about 360 nm to about 380 nm, about 360 nm to about 400 nm, or about 380 nm to about 400 nm. Extracted oils may have a residence time of UV exposure of no less than about 1 second, 5 seconds, 10 seconds, 20 seconds, 30 seconds, 1 minute, 5 minutes, 10 minutes, 30 minutes, 60 minutes, 2 hours, 3 hours, or no less than about 6 hours. Extracted oils may have a residence time for UV exposure of no more than about 6 hours, 3 hours, 2 hours, 60 minutes, 30 minutes, 10 minutes, 5 minutes, 1 minute, 30 seconds, 20 seconds, 10 seconds, 5 seconds, or no more than about 1 second.

In some aspects, a cannabinoid composition may be frozen after an extraction process. The freezing process may occur before, during, or after any post-extraction processes. A cannabinoid composition may be frozen as a method of extending shelf life. A cannabinoid composition may be frozen as part of a freeze-drying process.

In some cases, a cannabinoid composition may have a particular shelf-life. A shelf-life may be defined as the length of time at which one or more cannabinoid components within the cannabinoid composition maintain a desired level. For example, a shelf-life may be determined by the amount of time until the CBD level of a particular cannabinoid composition decreases by 50%. A shelf-life may also be determined by the length of time until an unwanted side product achieves a determined level in the cannabinoid composition. The shelf-life of a cannabinoid composition may be determined by chemical processes such as oxidation, or side-reactions between components and solvents. A particular cannabinoid composition may have a shelf-life of about 1 week, 2 weeks, 1 month, 3 months, 6 months, 1 year, 2 years, 5 years or more. A cannabinoid composition may have a shelf-life of at least about 1 week, 2 weeks, 1 month, 3 months, 6 months, 1 year, 2 years, 5 years or more. A cannabinoid composition may have a shelf-life of no more than about 5 years, 2 years, 1 year, 6 months, 3 months, 1 month, 2 weeks, 1 week or less. A particular cannabinoid extraction process or post-extraction process may increase the shelf-life of a cannabinoid composition by about 10%, 20%, 30%, 40%, 50%, 75%, 100%, 150%, 200% or more. A particular cannabinoid extraction process or post-extraction process may increase the shelf-life of a cannabinoid composition by at least about 10%, 20%, 30%, 40%, 50%, 75%, 100%, 150%, 200% or more. A particular cannabinoid extraction process or post-extraction process may increase the shelf-life of a cannabinoid composition by no more than about 200%, 150%, 100%, 75%, 50%, 40%, 30%, 20%, or 10% or less.

### Computer Systems

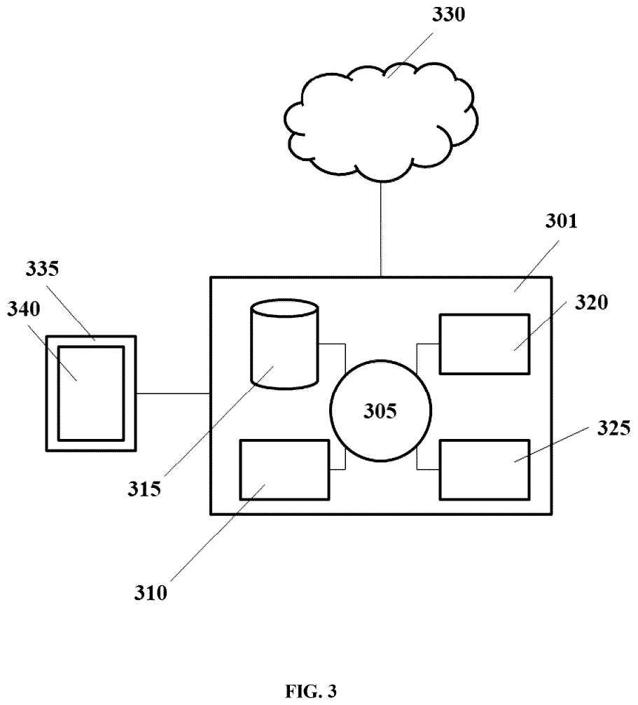

The present disclosure provides computer systems that are programmed to implement methods of the disclosure. FIG. 3 shows a computer system 301 that is programmed or otherwise configured to regulate various aspects of the plant processing methods of the present disclosure, such as, for example, the continuous processing of a Cannabis plant. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, instructions for the continuous processing of the Cannabis plant. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, automatically control the continuous processing method of the Cannabis plant.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-23. (canceled)

24. A modular system for a continuous processing of a Cannabis plant, the modular system comprising two or more processing units selected from the group consisting of:
(i) a foliage removal unit;
(ii) a de-gumming unit;
(iii) a trichome collection unit;
(iv) a seed collection unit;
(v) a seed processing unit;
(vi) a bast fiber collection unit;
(vii) a bast fiber processing unit;
(viii) a bundling residual biomass unit;
(ix) a cannabinoid extraction unit;
(x) a biomass gasification unit;
(xi) a biochar production unit; and
(xii) a biomass pelletization unit,
wherein said two or more additional processing units are operatively coupled to each other for said continuous processing of said Cannabis plant.

25. (canceled)

26. The modular system of claim 24, comprising three or more of said processing units selected from the group consisting of: (i)-(xii).

27. The modular system of claim 24, wherein said two or more additional processing units are operatively coupled to each other via one or more transport units.

28. The modular system of claim 24, wherein said one or more transport units is selected from the group consisting of: a roller, a belt, a chain, a chute, and a pulley.

29. The modular system of claim 24, wherein said two or more additional processing units are releasably coupled to each other.
